(19) United States
(12) Patent Application Publication
(10) Pub. No.: US 2024/0327369 A1
(43) Pub. Date: Oct. 3, 2024

(54) A METHOD FOR PREPARING HEXAHYDROCANNABINOL

(71) Applicant: BlackStone Therapeutics, LLC, Parker, CO (US)

(72) Inventors: Arianna C. COLLINS, Parker, CO (US); Kyle P. RAY, Parker, CO (US); Westley CRUCES, Parker, CO (US)

(21) Appl. No.: 18/290,868

(22) PCT Filed: Jul. 22, 2022
(86) PCT No.: PCT/US2022/074047

§ 371 Date: Jan. 22, 2024

Related U.S. Application Data
(60) Provisional application No. 63/225,287, filed on Jul. 23, 2021.
(60) Provisional application No. 63/299,278, filed on Jan. 13, 2022.

(51) Int. Cl. C07D 311/80; A61K 31/00

(52) U.S. Cl. CPC C07D 311/80; A61K 31/658

(57) ABSTRACT

Methods for producing phytocannabinoid derivatives having increased sp³ fraction and hexahydrocannabinol are disclosed. The methods can include hydrogenating the cyclohexenyl olefin group of various tetrahydro-phytocannabinoids in the presence of hydrogen gas, a hydrogen gas source, or a mixture thereof to produce the corresponding hexahydro-phytocannabinoid derivatives. The methods can include hydrogenating delta-8 tetrahydrocannabinol, delta-9 tetrahydrocannabinol, or a mixture thereof in the presence of hydrogen gas, a hydrogen gas source, or a mixture thereof to produce hexahydrocannabinol.

### BACKGROUND OF THE INVENTION

### I. Field of the Invention

This invention relates to the fields of organic chemistry and medicinal chemistry.

### II. Background

Cannabis products have recently garnered attention due to the high therapeutic potential of their cannabinoid content. Cannabinoids are compounds that bind to and activate cannabinoid receptors (CB1 and CB2) in the body. Cannabinoid receptors are present in neuronal cells in the brain and in numerous peripheral tissues throughout the body. Cannabinoid receptors are part of the body's endocannabinoid system (ECS) which plays a role in a number of physiological functions, including appetite, metabolism, pain, inflammation, mood, motor control, and sleep.

More than 90 different natural cannabinoids have been reported in the literature. The more well-known cannabinoids are tetrahydrocannabinol (THC), tetrahydrocannabinolic acid (THCA), cannabidiolic acid (CBDA), and cannabinolic acid (CBNA). In addition to the large number of natural cannabinoids, a number of synthetic and semi-synthetic cannabinoids have been identified. Like their natural counterparts, many synthetic and semi-synthetic cannabinoids have the potential to be used in therapeutic applications.

Over the last two decades, the endocannabinoid system (ECS) has emerged as target of pharmacotherapy of considerable physiological significance. This system includes two cannabinoid receptors (CB1 and CB2) and endogenous ligands named endocannabinoids. The identification of the CB1 and CB2 receptors together with the discovery of their endogenous ligands in the late 80s and early 90s, resulted in a major effort aimed at understanding the mechanisms and physiological roles of the ECS.

The ECS plays key modulatory roles during synaptic plasticity and homeostatic processes in the brain. Based on anecdotal evidence obtained from cannabis use, laboratory studies, and emerging clinical work, modulation of the ECS has been proposed as a promising therapeutic target to treat numerous central nervous system (CNS) disorders including neurodegenerative diseases, epilepsy, and cognitive deficits among others.

Endocannabinoids are physiologically occurring, biologically active compounds that bind to and activate CB1 and CB2 receptors with multiple physiological functions. Endocannabinoids have been found to have many physiological and patho-physiological functions, including mood alteration, control of feeding and appetite, motor and coordination activities, analgesia, immune modulation, and gut motility.

Phytocannabinoids are a structurally diverse class of naturally occurring chemical constituents in the Cannabis sativa plant. Of the many phytocannabinoids, Δ9-THC and cannabidoil (CBD) have garnered the most interest. Δ9-THC is responsible for the psychoactive effects of Cannabis sativa mediated by the activation of CB1 receptor in the brain, whereas CBD is considered non-psychotropic. Recently, these compounds have generated considerable interest due to their beneficial neuroprotective, antiepileptic, anxiolytic, antipsychotic, and anti-inflammatory properties. Drug discovery programs in both industry and academia have sought to improve the potency, efficacy, and/or pharmacokinetic properties of these interesting phytocannabinoids. The THC/CBD scaffold is becoming a target of increasing interest for medicinal chemists for providing novel, synthetic alternatives to THC and CBD.

### SUMMARY OF THE INVENTION

One semi-synthetic cannabinoid is hexahydrocannabinol (HHC). HHC is structurally similar to THC, and it has been shown to have many of the same useful therapeutic properties. The present disclosure provides a novel means for producing HHC from relatively abundant and inexpensive starting materials.

Some aspects of the disclosure are therefore directed to a method for producing hexahydrocannabinol. In some embodiments, the method comprises providing a starting composition comprising delta-8 tetrahydrocannabinol, delta-9 tetrahydrocannabinol, or a mixture thereof to a reaction vessel, providing a catalyst to the reaction vessel, providing hydrogen gas, a source of hydrogen gas, or a combination thereof to the reaction vessel, and heating the reaction vessel to a temperature sufficient to effect hydrogenation of the delta-8 tetrahydrocannabinol, delta-9 tetrahydrocannabinol, or mixture thereof. In some embodiments, a mixture of delta-8 tetrahydrocannabinol and delta-9 tetrahydrocannabinol is obtained by cyclization reaction of cannabidiol (CBD). In some embodiments, the delta-8 tetrahydrocannabinol and/or delta-9 tetrahydrocannabinol aromatic ring are not affected by hydrogenation, therefore, only the non-aromatic olefin is hydrogenated. In some embodiments, hydrogenation of delta-8 tetrahydrocannabinol, delta-9 tetrahydrocannabinol, or a mixture thereof produces hexahydrocannabinol. In some embodiments, the methods disclosed herein can be used to produce hexahydrocannabinol from other cannabinol derivatives that include a non-aromatic olefin in the cyclohexyl ring opposite the aromatic ring, e.g., delta-10 tetrahydrocannabinol. In some embodiments, hexahydrocannabinol produced by the methods disclosed herein contains less than 0.3% by weight of delta-9 THC.

In some embodiments, the catalyst is provided in an amount ranging from 0.01 to 10 molar equivalents, based on the amount of delta-8 tetrahydrocannabinol, delta-9 tetrahydrocannabinol, or the combined amount of delta-8 tetrahydrocannabinol and delta-9 tetrahydrocannabinol. In some embodiments, the catalyst is provided in an amount ranging from 0.1 to 5 molar equivalents, based on the amount of delta-8 tetrahydrocannabinol, delta-9 tetrahydrocannabinol, or the combined amount of delta-8 tetrahydrocannabinol and delta-9 tetrahydrocannabinol. In some embodiments, the catalyst comprises a metal selected from the group consisting of palladium, rhodium, nickel, aluminum, platinum, and iridium. In some embodiments, the catalyst is provided on a support. In some embodiments, the catalyst is selected from the group consisting of Pd/C, Rh/C. Pt/C, Ru/C. Raney nickel. palladium on alumina, palladium on activated charcoal, Pt₂O (Adam's catalyst), [C₈H₁₂IrP(C₆H₁₁)₃C₅H₅N]PF₆ (Wilkinson's catalyst), and [RhCl(PPh₃)₃] (Crabtree's catalyst). In some embodiments, the hydrogen gas is provided in an amount that affords an intra-vessel gas pressure ranging from 1 bar to 20 bar. In some embodiments, the hydrogen gas is provided in an amount that affords an intra-vessel gas pressure ranging from 1 bar to 5 bar. In some embodiments, hydrogen gas is not provided to the reaction vessel. In some embodiments, the source of hydrogen gas generates hydrogen gas in situ. In some embodiments, the source of hydrogen gas comprises ammonium formate and formic acid. In some embodiments, when ammonium formate and formic acid are combined in the presence of a catalyst, the formic acid will decompose into carbon dioxide and hydrogen gas; the hydrogen gas generated by this decomposition reaction can be used to hydrogenate delta-8 tetrahydrocannabinol, delta-9 tetrahydrocannabinol, or a mixture thereof. In some embodiments, an amount of ammonium formate ranges from 1 to 40 molar equivalents, based on the amount of delta-8 tetrahydrocannabinol, delta-9 tetrahydrocannabinol, or the combined amount of delta-8 tetrahydrocannabinol and delta-9 tetrahydrocannabinol. In some embodiments, an amount of ammonium formate ranges from 5 to 20 molar equivalents, based on the amount of delta-8 tetrahydrocannabinol, delta-9 tetrahydrocannabinol, or the combined amount of delta-8 tetrahydrocannabinol and delta-9 tetrahydrocannabinol. In some embodiments, an amount of formic acid ranges from 1 to 40 molar equivalents, based on the amount of delta-8 tetrahydrocannabinol, delta-9 tetrahydrocannabinol, or the combined amount of delta-8 tetrahydrocannabinol and delta-9 tetrahydrocannabinol. In some embodiments, an amount of formic acid ranges from 5 to 20 molar equivalents, based on the amount of delta-8 tetrahydrocannabinol, delta-9 tetrahydrocannabinol, or the combined amount of delta-8 tetrahydrocannabinol and delta-9 tetrahydrocannabinol.

In some embodiments, a method for producing hexahydrocannabinol further comprises providing a solvent to the reaction vessel prior to the heating step. In some embodiments, the solvent is selected from the group consisting of ethanol, methanol, propanol, isopropanol, butanol, sec-butanol, and isobutanol. In some embodiments, the solvent is a polar protic solvent known to those of skill in the art. In some embodiments, hydrogenation of delta-8 tetrahydrocannabinol hydrogenates the delta-8 olefin. In some embodiments, hydrogenation of delta-9 tetrahydrocannabinol hydrogenates the delta-9 olefin. In some embodiments, hydrogenation of a mixture of delta-8 tetrahydrocannabinol and delta-9 tetrahydrocannabinol hydrogenates the delta-8 olefin of delta-8 tetrahydrocannabinol and hydrogenates the delta-9 olefin of delta-9 tetrahydrocannabinol. Therefore, in some embodiments, hydrogenation of either delta-8 tetrahydrocannabinol, delta-9 tetrahydrocannabinol or both delta-8 tetrahydrocannabinol and delta-9 tetrahydrocannabinol produces hexahydrocannabinol.

In some embodiments, the heating step comprises heating the reaction vessel to a temperature ranging from 25° C. to 100° C. In some embodiments, the reaction vessel interior is hermetically sealed. In some embodiments, the reaction vessel interior is hermetically sealed prior to addition of any components, including solvent, starting composition, hydrogen gas, catalyst, or source of hydrogen gas. In some embodiments, the reaction is purged with an inert gas prior to addition of starting composition, catalyst, and hydrogen gas, source of hydrogen gas, or combination thereof. In some embodiments, the inert gas is nitrogen or argon.

In some embodiments, a method for producing hexahydrocannabinol comprises the steps of providing a starting composition comprising a mixture of delta-8 tetrahydrocannabinol and delta-9 tetrahydrocannabinol to a reaction vessel, providing Pd/C and hydrogen gas to the reaction vessel, and heating the reaction vessel to a temperature sufficient to effect hydrogenation of the delta-8 tetrahydrocannabinol, delta-9 tetrahydrocannabinol, or mixture thereof to produce hexahydrocannabinol.

In some embodiments, a method for producing hexahydrocannabinol comprises the steps of providing a starting composition comprising a mixture of delta-8 tetrahydrocannabinol and delta-9 tetrahydrocannabinol to a reaction vessel, providing Pd/C, ammonium formate, and formic acid to the reaction vessel, and heating the reaction vessel to a temperature sufficient to effect hydrogenation of the delta-8 tetrahydrocannabinol, delta-9 tetrahydrocannabinol, or mixture thereof to produce hexahydrocannabinol.

In some embodiments, a method for producing hexahydrocannabinol comprises the steps of providing a starting composition comprising a mixture of delta-8 tetrahydrocannabinol and delta-9 tetrahydrocannabinol to a reaction vessel, providing Pd/C, hydrogen gas, ammonium formate, and formic acid to the reaction vessel, and heating the reaction vessel to a temperature sufficient to effect hydrogenation of the delta-8 tetrahydrocannabinol, delta-9 tetrahydrocannabinol, or mixture thereof to produce hexahydrocannabinol.

Some aspects of the disclosure are directed to a process for the preparation of a hexahydrocannabidiol derivative. In some aspects, the process comprises the steps of providing a tetrahydrocannabidiol derivative of formula I to a reaction vessel;

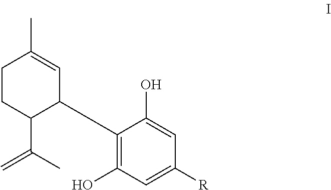

wherein R is hydrogen or a substituted or unsubstituted alkyl group ranging from 1 to 9 carbon atoms; providing a catalyst to the reaction vessel; providing hydrogen gas, a source of hydrogen gas, or a combination thereof to the reaction vessel; and heating the reaction vessel to a temperature sufficient to effect hydrogenation of the cyclohexenyl olefin group to produce a hexahydrocannabidiol derivative of formula II

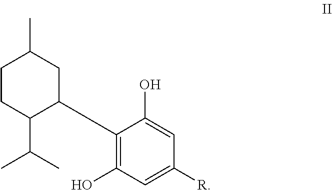

In some aspects, the process comprises the steps of providing a tetrahydrocannabidiol derivative of formula I to a reaction vessel;

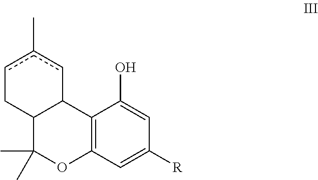

wherein R is hydrogen or a substituted or unsubstituted alkyl group ranging from 1 to 9 carbon atoms; providing a catalyst to the reaction vessel; providing hydrogen gas, a source of hydrogen gas, or a combination thereof to the reaction vessel; and heating the reaction vessel to a temperature sufficient to effect hydrogenation of the cyclohexenyl olefin group to produce a hexahydrocannabinoid derivative of formula IV

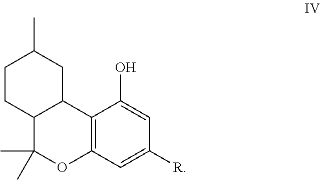

In some aspects, R is propyl or heptyl. In some aspects, R is CF₃, —CH₂F, —(CH₂)₂F, —(CH₂)₃F, —(CH₂)₄F, —(CH₂)₅F, —(CH₂)₆F, —(CH₂)₇F, or —(CH₂)₇F, or —(CH₂)₈F. In some aspects, the catalyst is provided in an amount ranging from 0.01 to 10 molar equivalents. The catalyst can be provided at any one of, less than, greater than, between, or any range thereof of 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 molar equivalents, such as such as 0.02 molar equivalents to 0.20 molar equivalents. In some aspects, the catalyst is selected from the group consisting of Pd/C, Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([C₈H₁₂IrP(C₆H₁₁)₃C₅H₅N]PF₆), 9-borabicyclo[3.3.1]nonane, alpine borane, BH₃-DMSO, BH₃-THF, and N-methylimidodiacetic (MIDA) boronates. In certain aspects, the catalyst is Pd/C. In some aspects, the hydrogen gas is provided in an amount that affords an intra-vessel gas pressure ranging from 1 bar to 20 bar. The hydrogen gas can be provided at any one of, less than, greater than, between, or any range thereof of 1, 2, 3, 4, 5, 6, 7, 8, 9, 10, 11, 12, 13, 14, 15, 16, 17, 18, 19, or 20 bar, such as 5 bar to 10 bar. In some aspects, hydrogen gas is not provided to the reaction vessel. In some aspects, the source of hydrogen gas generates hydrogen gas in situ. In some aspects, the source of hydrogen gas comprises ammonium formate and/or formic acid. In some aspects, an amount of ammonium formate ranges from 1 to 40 molar equivalents. The amount of ammonium formate can be provided at any one of, less than, greater than, between, or any range thereof of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 molar equivalents, such as 10 molar equivalents to 20 molar equivalents. In some aspects, an amount of formic acid ranges from 1 to 40 molar equivalents. The amount of formic acid can be provided at any one of, less than, greater than, between, or any range thereof of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 molar equivalents, such as 1 molar equivalents to 5 molar equivalents. In some aspects, the process further comprises the step of providing a solvent to the reaction vessel prior to the heating step. In some aspects, the solvent is selected from the group consisting of methanol, ethanol, propanol, isopropanol, butanol, tert-butanol, THF, 2-Me-THF, toluene, and ethyl acetate. In some aspects, the heating step comprises heating the reaction vessel to a temperature ranging from 25° C. to 100° C. The temperature can be any one of, less than, greater than, between, or any range thereof of 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C., such as 50° C. to 60° C. In some aspects, the reaction vessel is hermetically sealed. In some aspects, the process further comprises the step of purging the reaction vessel with an inert gas prior to addition of reactants and catalyst. In some aspects, the inert gas is nitrogen or argon.

Some aspects of the disclosure are directed to a process for the preparation of hexahydrocannabidivarin (HCBDV). In some aspects, the process comprises providing cannabidivarin (CBDV) to a reaction vessel: providing a catalyst to the reaction vessel; providing hydrogen gas, a source of hydrogen gas, or a combination thereof to the reaction vessel; and heating the reaction vessel to a temperature sufficient to effect hydrogenation of the CBDV cyclohexenyl olefin group to produce HCBDV.

Some aspects of the disclosure are directed to a process for the preparation of hexahydrocannabidiphorol (HCBDP). In some aspects, the process comprises providing cannabidiphorol (CBDP) to a reaction vessel; providing a catalyst to the reaction vessel; providing hydrogen gas, a source of hydrogen gas, or a combination thereof to the reaction vessel; and heating the reaction vessel to a temperature sufficient to effect hydrogenation of the CBDP cyclohexenyl olefin group to produce HCBDP.

Some aspects of the disclosure are directed to a process for the preparation of hexahydrocannabivarin (HHCV). In some aspects, the process comprises providing tetrahydrocannabivarin (THCV) to a reaction vessel; providing a catalyst to the reaction vessel; providing hydrogen gas, a source of hydrogen gas, or a combination thereof to the reaction vessel; and heating the reaction vessel to a temperature sufficient to effect hydrogenation of the THCV cyclohexenyl olefin group to produce HHCV.

Some aspects of the disclosure are directed to a process for the preparation of hexahydrocannabiphorol (HHCP). In some aspects, the process comprises providing tetrahydrocannabiphorol (THCP) to a reaction vessel; providing a catalyst to the reaction vessel; providing hydrogen gas, a source of hydrogen gas, or a combination thereof to the reaction vessel; and heating the reaction vessel to a temperature sufficient to effect hydrogenation of the THCP cyclohexenyl olefin group to produce HHCP.

Some aspects of the disclosure are directed to a compound of formula II:

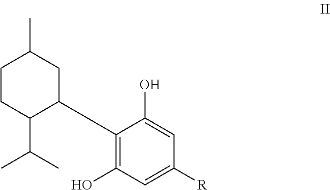

wherein R is hydrogen or a substituted or unsubstituted alkyl group ranging from 1 to 9 carbon atoms. In some aspects, the compound is further defined as

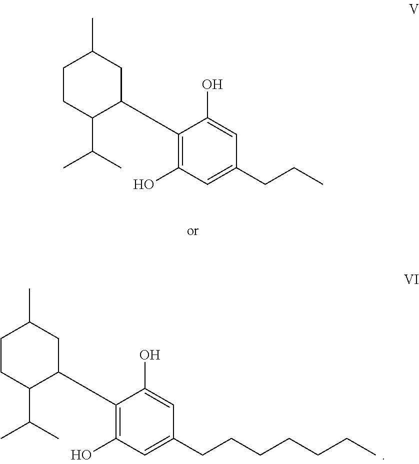 [SMILES: CCCc1cc(O)c(C2CC(C)CCC2C(C)C)c(O)c1 ; CCCCCCCc1cc(O)c(C2CC(C)CCC2C(C)C)c(O)c1]

Some aspects of the disclosure are directed to a compound of formula IV

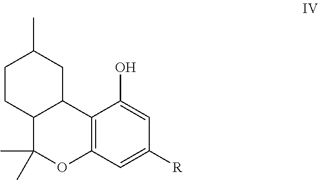

wherein R is hydrogen or a substituted or unsubstituted alkyl group ranging from 1 to 9 carbon atoms. In some aspects, the compound is further defined as

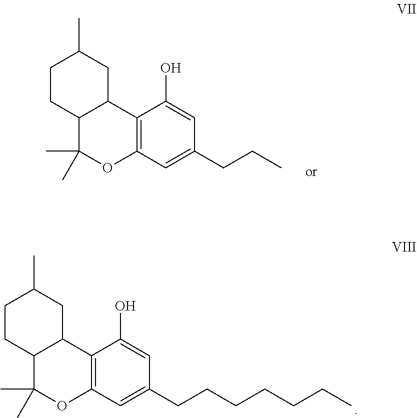

Some aspects of the disclosure are directed to a pharmaceutical composition comprising a compound as disclosed herein. The terms delta-8 tetrahydrocannabinol, delta-8 THC, D8 THC, Δ8-tetrahydrocannabinol, and Δ8-THC are used interchangeably herein. The terms delta-9 tetrahydrocannabinol, delta-9 THC, D9 THC, Δ9-tetrahydrocannabinol, Δ9-THC, and THC are used interchangeably herein. The terms hexahydrocannabinol and HHC are used interchangeably herein. The term “semi-synthetic” is defined as a method that employs natural compounds or compounds derived from natural compounds as starting materials to produce different compounds.

In the context of the present invention, at least the following 67 aspects are described. Aspect 1 is a method for producing hexahydrocannabinol, comprising providing a starting composition comprising delta-8 tetrahydrocannabinol, delta-9 tetrahydrocannabinol, or a mixture thereof to a reaction vessel; providing a catalyst to the reaction vessel; providing hydrogen gas, a source of hydrogen gas, or a combination thereof to the reaction vessel; heating the reaction vessel to a temperature sufficient to effect hydrogenation of the delta-8 tetrahydrocannabinol, delta-9 tetrahydrocannabinol, or mixture thereof; wherein hydrogenation of the delta-8 tetrahydrocannabinol, delta-9 tetrahydrocannabinol, or mixture thereof produces hexahydrocannabinol. Aspect 2 method of Aspect 1, wherein the catalyst is provided in an amount ranging from 0.01 to 10 molar equivalents. Aspect 3 is the method of Aspect 2, wherein the catalyst comprises a metal selected from the group consisting of palladium, rhodium, nickel, aluminum, platinum, and iridium. Aspect 4 is the method of Aspect 3, wherein the catalyst is selected from the group consisting of Pd/C, Rh/C, Pt/C, Ru/C, Rancy nickel. palladium on alumina, palladium on activated charcoal, Pt₂O, [C₈H₁₂IrP(C₆H₁₁)₃C₅H₅N]PF₆, and [RhCl(PPh₃)₃]. Aspect 5 is the method of Aspect 1, wherein the hydrogen gas is provided in an amount that affords an intra-vessel gas pressure ranging from 1 bar to 20 bar. Aspect 6 is the method of Aspect 1, wherein hydrogen gas is not provided to the reaction vessel. Aspect 7 is the method of Aspect 1, wherein the source of hydrogen gas generates hydrogen gas in situ. Aspect 8 is the method of Aspect 1, wherein the source of hydrogen gas comprises ammonium formate and formic acid. Aspect 9 is the method of Aspect 8, wherein an amount of ammonium formate ranges from 1 to 40 molar equivalents. Aspect 10 is the method of Aspect 8, wherein an amount of formic acid ranges from 1 to 40 molar equivalents. Aspect 11 is the method of Aspect 1, further comprising providing a solvent to the reaction vessel prior to the heating step. Aspect 12 is the method of Aspect 1, wherein the solvent is selected from the group consisting of ethanol, methanol, propanol, isopropanol, butanol, sec-butanol, and isobutanol. Aspect 13 is the method of Aspect 1, wherein hydrogenation of the delta-8 tetrahydrocannabinol hydrogenates the delta-8 olefin. Aspect 14 is the method of Aspect 1, wherein hydrogenation of the delta-9 tetrahydrocannabinol hydrogenates the delta-9 olefin. Aspect 15 is the method of Aspect 1, wherein hydrogenation of the mixture of delta-8 tetrahydrocannabinol and delta-9 tetrahydrocannabinol hydrogenates the delta-8 olefin of delta-8 tetrahydrocannabinol and hydrogenates the delta-9 olefin of delta-9 tetrahydrocannabinol. Aspect 16 is the method of Aspect 1, wherein the heating step comprises heating the reaction vessel to a temperature ranging from 25° C. to 100° C. Aspect 17 is the method of Aspect 1, wherein the reaction vessel is hermetically sealed. Aspect 18 is the method of Aspect 1, further comprising purging the reaction vessel with an inert gas prior to addition of reactants and catalyst. Aspect 19 is the method of Aspect 18, wherein the inert gas is nitrogen or argon. Aspect 20 is a method for producing hexahydrocannabinol, comprising providing a starting composition comprising a mixture of delta-8 tetrahydrocannabinol and delta-9 tetrahydrocannabinol to a reaction vessel; providing Pd/C and hydrogen gas to the reaction vessel; and heating the reaction vessel to a temperature sufficient to effect hydrogenation of the delta-8 tetrahydrocannabinol, delta-9 tetrahydrocannabinol, or mixture thereof; wherein hydrogenation of the delta-8 tetrahydrocannabinol and delta-9 tetrahydrocannabinol produces hexahydrocannabinol. Aspect 21 is a method for producing hexahydrocannabinol, comprising providing a starting composition comprising a mixture of delta-8 tetrahydrocannabinol and delta-9 tetrahydrocannabinol to a reaction vessel; providing Pd/C, ammonium formate, and formic acid to the reaction vessel; and heating the reaction vessel to a temperature sufficient to effect hydrogenation of the delta-8 tetrahydrocannabinol, delta-9 tetrahydrocannabinol, or mixture thereof; wherein hydrogenation of the delta-8 tetrahydrocannabinol and delta-9 tetrahydrocannabinol produces hexahydrocannabinol. Aspect 22 is a method for producing hexahydrocannabinol, comprising providing a starting composition comprising a mixture of delta-8 tetrahydrocannabinol and delta-9 tetrahydrocannabinol to a reaction vessel; providing Pd/C, hydrogen gas, ammonium formate, and formic acid to the reaction vessel; and heating the reaction vessel to a temperature sufficient to effect hydrogenation of the delta-8 tetrahydrocannabinol, delta-9 tetrahydrocannabinol, or mixture thereof; wherein hydrogenation of the delta-8 tetrahydrocannabinol and delta-9 tetrahydrocannabinol produces hexahydrocannabinol. Aspect 23 is a process for the preparation of a hexahydrocannabidiol derivative, comprising: providing a tetrahydrocannabidiol derivative of formula I to a reaction vessel;

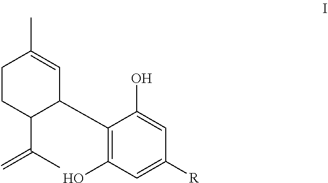

wherein R is hydrogen or a substituted or unsubstituted alkyl group ranging from 1 to 9 carbon atoms; providing a catalyst to the reaction vessel; providing hydrogen gas, a source of hydrogen gas, or a combination thereof to the reaction vessel; and heating the reaction vessel to a temperature sufficient to effect hydrogenation of the cyclohexenyl olefin group to produce a hexahydrocannabidiol derivative of formula II

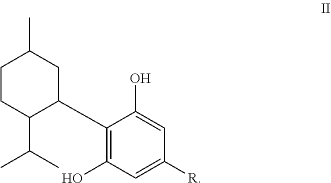

Aspect 24 is the process of Aspect 23, wherein R is propyl or heptyl. Aspect 25 is the process of Aspect 23, wherein the catalyst is provided in an amount ranging from 0.01 to 10 molar equivalents. Aspect 26 is the process of Aspect 25, wherein the catalyst is selected from the group consisting of Pd/C, Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([C₈H₁₂IrP(C₆H₁₁)₃C₅H₅N]PF₆), 9-borabicyclo[3.3.1]nonane, alpine borane, BH₃-DMSO, BH₃-THF, and N-methylimidodiacetic (MIDA) boronates. Aspect 27 is the process of Aspect 26, wherein the catalyst is Pd/C. Aspect 28 is the process of Aspect 23, wherein the hydrogen gas is provided in an amount that affords an intra-vessel gas pressure ranging from 1 bar to 20 bar. Aspect 29 is the process of Aspect 23, wherein hydrogen gas is not provided to the reaction vessel. Aspect 30 is the process of Aspect 23, wherein the source of hydrogen gas generates hydrogen gas in situ. Aspect 31 is the process of Aspect 23, wherein the source of hydrogen gas comprises ammonium formate and/or formic acid. Aspect 32 is the process of Aspect 31, wherein an amount of ammonium formate ranges from 1 to 40 molar equivalents. Aspect 33 is the process of Aspect 31, wherein an amount of formic acid ranges from 1 to 40 molar equivalents. Aspect 34 is the process of Aspect 23, further comprising providing a solvent to the reaction vessel prior to the heating step. Aspect 35 is the process of Aspect 23, wherein the solvent is selected from the group consisting of methanol, ethanol, propanol, isopropanol, butanol, tert-butanol, THF, 2-Me-THF, toluene, and ethyl acetate. Aspect 36 is the process of Aspect 23, wherein the heating step comprises heating the reaction vessel to a temperature ranging from 25° C. to 100° C. Aspect 37 is the process of Aspect 23, wherein the reaction vessel is hermetically sealed. Aspect 38 is the process of Aspect 23, further comprising purging the reaction vessel with an inert gas prior to addition of reactants and catalyst. Aspect 39 is the process of Aspect 38, wherein the inert gas is nitrogen or argon. Aspect 40 is a process for the preparation of a hexahydrocannabinoid derivative, comprising providing a tetrahydrocannabinoid derivative of formula III to a reaction vessel;

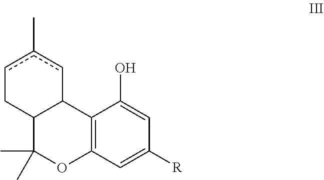

wherein R is hydrogen or a substituted or unsubstituted alkyl group ranging from 1 to 9 carbon atoms; providing a catalyst to the reaction vessel; providing hydrogen gas, a source of hydrogen gas, or a combination thereof to the reaction vessel; and heating the reaction vessel to a temperature sufficient to effect hydrogenation of the cyclohexenyl olefin group to produce a hexahydrocannabinoid derivative of formula IV

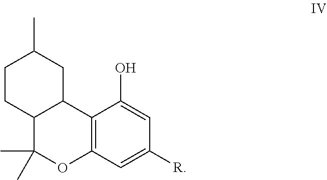

Aspect 41 is the process of Aspect 40, wherein R is propyl or heptyl. Aspect 42 is the process of Aspect 40, wherein the catalyst is provided in an amount ranging from 0.01 to 10 molar equivalents. Aspect 43 is the process of Aspect 42, wherein the catalyst is selected from the group consisting of Pd/C, Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([C₈H₁₂IrP(C₆H₁₁)₃C₅H₅N]PF₆), 9-borabicyclo[3.3.1]nonane, alpine borane, BH₃-DMSO, BH₃-THF, and N-methylimidodiacetic (MIDA) boronates. Aspect 44 is the process of Aspect 43, wherein the catalyst is Pd/C. Aspect 45 is the process of Aspect 40, wherein the hydrogen gas is provided in an amount that affords an intra-vessel gas pressure ranging from 1 bar to 20 bar. Aspect 46 is the process of Aspect 40, wherein hydrogen gas is not provided to the reaction vessel. Aspect 47 is the process of Aspect 40, wherein the source of hydrogen gas generates hydrogen gas in situ. Aspect 48 is the process of Aspect 40, wherein the source of hydrogen gas comprises ammonium formate and/or formic acid. Aspect 49 is the process of Aspect 48, wherein an amount of ammonium formate ranges from 1 to 40 molar equivalents. Aspect 50 is the process of Aspect 48, wherein an amount of formic acid ranges from 1 to 40 molar equivalents. Aspect 51 is the process of Aspect 40, further comprising providing a solvent to the reaction vessel prior to the heating step. Aspect 52 is the process of Aspect 40, wherein the solvent is selected from the group consisting of methanol, ethanol, propanol, isopropanol, butanol, tert-butanol, THF, 2-Me-THF, toluene, and ethyl acetate. Aspect 53 is the process of Aspect 40, wherein the heating step comprises heating the reaction vessel to a temperature ranging from 25° C. to 100° C. Aspect 54 is the process of Aspect 40, wherein the reaction vessel is hermetically sealed. Aspect 55 is the process of Aspect 40, further comprising purging the reaction vessel with an inert gas prior to addition of reactants and catalyst. Aspect 56 is the process of Aspect 55, wherein the inert gas is nitrogen or argon. Aspect 57 is a process for the preparation of hexahydrocannabidivarin (HCBDV), comprising providing cannabidivarin (CBDV) to a reaction vessel; providing a catalyst to the reaction vessel; providing hydrogen gas, a source of hydrogen gas, or a combination thereof to the reaction vessel; and heating the reaction vessel to a temperature sufficient to effect hydrogenation of the CBDV cyclohexenyl olefin group to produce HCBDV. Aspect 58 is a process for the preparation of hexahydrocannabidiphorol (HCBDP), comprising providing cannabidiphorol (CBDP) to a reaction vessel; providing a catalyst to the reaction vessel; providing hydrogen gas, a source of hydrogen gas, or a combination thereof to the reaction vessel; and heating the reaction vessel to a temperature sufficient to effect hydrogenation of the CBDP cyclohexenyl olefin group to produce HCBDP. Aspect 59 is a process for the preparation of hexahydrocannabivarin (HHCV), comprising providing tetrahydrocannabivarin (THCV) to a reaction vessel; providing a catalyst to the reaction vessel; providing hydrogen gas, a source of hydrogen gas, or a combination thereof to the reaction vessel; and heating the reaction vessel to a temperature sufficient to effect hydrogenation of the THCV cyclohexenyl olefin group to produce HHCV. Aspect 60 is a process for the preparation of hexahydrocannabiphorol (HHCP), comprising providing tetrahydrocannabiphorol (THCP) to a reaction vessel; providing a catalyst to the reaction vessel; providing hydrogen gas, a source of hydrogen gas, or a combination thereof to the reaction vessel; and heating the reaction vessel to a temperature sufficient to effect hydrogenation of the THCP cyclohexenyl olefin group to produce HHCP. Aspect 61 is a compound of formula II

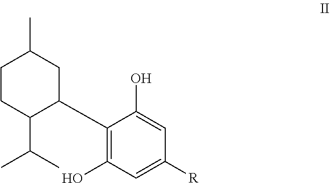

wherein R is hydrogen or a substituted or unsubstituted alkyl group ranging from 1 to 9 carbon atoms. Aspect 62 is the compound of Aspect 61, wherein the compound is further defined as

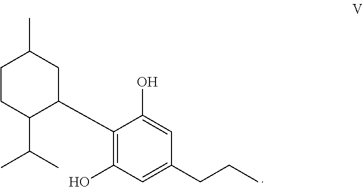 [SMILES: CCCc1cc(O)c(C2CC(C)CCC2C(C)C)c(O)c1]

Aspect 63 is the compound of Aspect 61, wherein the compound is further defined as

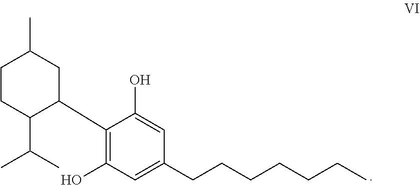 [SMILES: CCCCCCCc1cc(O)c(C2CC(C)CCC2C(C)C)c(O)c1]

Aspect 64 is a compound of formula IV:

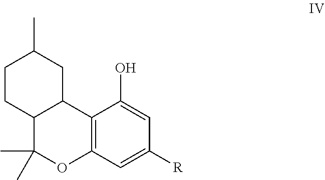

wherein R is hydrogen or a substituted or unsubstituted alkyl group ranging from 1 to 9 carbon atoms. Aspect 65 is the compound of Aspect 64, wherein the compound is further defined as

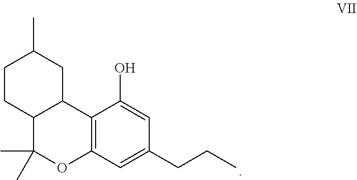 [SMILES: CCCc1cc(O)c2c(c1)OC(C)(C)C1CCC(C)CC21]

Aspect 66 is the compound of Aspect 64, wherein the compound is further defined as

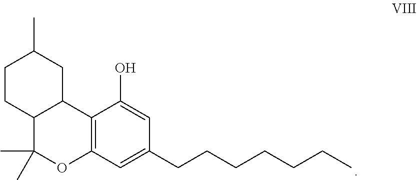 [SMILES: CCCCCCCc1cc(O)c2c(c1)OC(C)(C)C1CCC(C)CC21]

Aspect 67 is a pharmaceutical composition comprising a compound of any of Aspects 61 to 66.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B are reaction schemes that depicts the HHC product that is obtained by hydrogenation of the delta-8 olefin of delta-8 THC (FIG. 1A) and by hydrogenation of the delta-9 olefin of delta-9 THC (FIG. 1B).

FIG. 2 is a ¹H NMR spectra of the HHC product obtained by the method disclosed herein.

FIG. 3 is a 13C NMR spectra of the HHC product obtained by the method disclosed herein.

FIG. 4 is a gas chromatogram trace obtained from GC/MS analysis of the HHC product.

FIGS. 5A-5B depict mass spectrometry data obtained from GC/MS analysis of the HHC product. FIG. 5A includes data corresponding to the left peak of the GC trace, and includes prominent ions at m/z 193, 273, and 260. FIG. 5B includes data corresponding to the right peak of the LC trace, and includes prominent ions at m/z 193, 273, and 260.

FIG. 6 is a mass spectrum of HHC from the National Institute of Standards and Technology (NIST) database, and includes prominent ions at m/z 193, 273, and 260.

FIGS. 7A-7E are exemplary reaction schemes that depict products obtained by hydrogenation of the delta-8 olefin or delta-9 olefin of different phytocannabinoids. FIG. 7A hydrogenation of cannabidoil (CBD). FIG. 7B hydrogenation of cannabidivarin (CBDV).

FIG. 7C hydrogenation of cannabidiphorol (CBDP). FIG. 7D hydrogenation of tetrahydrocannabivarin (THCV). FIG. 7E hydrogenation of tetrahydrocannabiphorol (THCP).

FIG. 8 is an HPLC trace of the hexahydrocannabidiol (HCDB) product obtained by the method disclosed herein.

FIG. 9 is a ¹H NMR spectra of the HCBD product obtained by the method disclosed herein.

FIGS. 10A-10B 1H NMR spectra at different temperatures. FIG. 10A ¹H NMR of the HCBD product obtained by the method disclosed herein performed at 25° C. FIG. 10B 1H NMR of the HCBD product obtained by the method disclosed herein performed at 60° C.

FIG. 11 is a correlated spectroscopy (COSY) NMR spectra of the HCDB product obtained by the method disclosed herein.

FIG. 12 is an HPLC trace of CBDV starting material.

FIG. 13 is an HPLC trace of the hexahydrocannabidivarin (HCBDV) product obtained by the method disclosed herein.

FIG. 14 is an HPLC trace of CBDP starting material.

FIG. 15 is an HPLC trace of the hexahydrocannabidiphorol (HCBDP) product obtained by the method disclosed herein.

FIG. 16 is a ¹H NMR spectra of the HCBDP product obtained by the method disclosed herein.

FIG. 17 is a heteronuclear single quantum coherence (HSQC) NMR spectra of the HCBDP product obtained by the method disclosed herein.

FIG. 18 is an HPLC trace of THCV starting material.

FIG. 19 is an HPLC trace of the hexahydrocannabivarin (HHCV) product obtained by the method disclosed herein.

FIG. 20 is an HPLC trace of the hexahydrocannabivarin (HHCV) product oil obtained by the method disclosed herein.

FIG. 21 is a ¹H NMR spectra of the HHCV product obtained by the method disclosed herein.

FIG. 22 is a 13C NMR spectra of the HHCV product obtained by the method disclosed herein.

FIG. 23 is a correlated spectroscopy (COSY) NMR spectra of the HCDV product obtained by the method disclosed herein.

FIG. 24 is a heteronuclear single quantum coherence (HSQC) NMR spectra of the HHCV product obtained by the method disclosed herein.

FIG. 25 is an HPLC trace of THCP starting material.

FIG. 26 is an HPLC trace of the hexahydrocannabiphorol (HHCP) product obtained by the method disclosed herein.

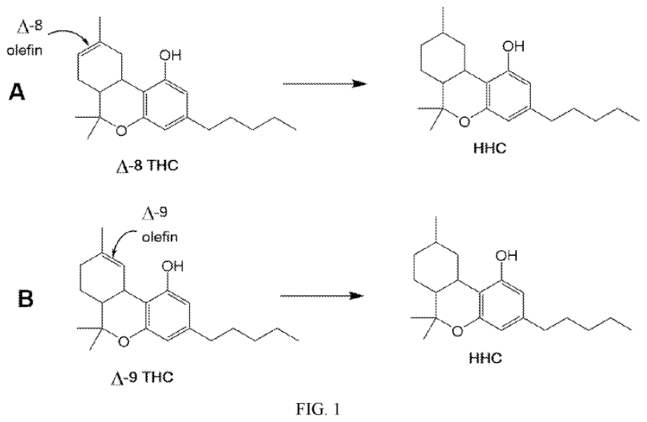

### DETAILED DESCRIPTION OF THE INVENTION

Cannabinoid use as medical therapy to treat diseases or alleviate symptoms has increased in recent years. Many consumers who do not want to use traditional cannabis products or those who live in places where cannabis products are not legally available are looking for alternative means to relieve stress and anxiety. Although many cannabinoids have similar structures, minor structural differences result in distinct ligand-receptor interactions. This allosteric regulation brings about downstream physiological effects that are different from effects triggered the parent compound, THC. Hexahydrocannabinol is structurally-related to THC, however, the differences in structure between THC and hexahydrocannabinol result in different effects that make hexahydrocannabinol an attractive alternative.

Understanding cannabinoid pharmacology centers around understanding molecular interactions between the cannabinoids and their molecular targets, cannabinoid receptor 1 (CB1) and cannabinoid receptor 2 (CB2). CBD is the second most abundant phytocannabinoid present in cannabis and accounts for up to 40% of dry mass in some cultivars. It is a partial agonist of the CB2 receptor, and can bind to other, non-cannabinoid receptors. Preliminary clinical data suggest that CBD may ameliorate the symptoms of anxiety, cognitive and movement disorders, pain, and epileptic seizures.

One of the parameters that can affect the biological activity of THC-like cannabinoids is the length of the alkyl chain. THCV is structurally similar to THC, with the only difference being two fewer carbons in the carbon tail: both molecules share similar traits, binding affinities, and metabolic derivatives. However, unlike THC, THCV has proposed anti-obesity activity. Tetrahydrocannabutol (THCB) and Cannabidibutol are phytocannabinoids with linear alkyl side chains containing four carbon atoms. Tetrahydrocannabiphorol (THCP) and Cannabidiphorol (CBDP) both include C7 linear alkyl side chains. These compounds have different chemical structures, which likely affects their receptor subtype selectivity.

As clinical reports on phytocannabinoid activity continue to be released, it is essential to understand how different structural variations affect binding affinity and physiological activity. Recent literature in medicinal chemistry has proposed simple measures to establish complex molecules as potential drug candidates. Increasing sp³ fraction (the number of sp³ carbons/total carbon count) and whether a chiral carbon exists in a molecule have been reported to correlate with success as compounds move from discovery to clinical testing.

As such, the present inventors have developed methods for the synthesis of new phytocannabinoid derivatives with variable alkyl chain length and increased sp³ fraction. These novel compounds include an aromatic ring, but are devoid of non-aromatic olefin groups. These compounds will provide insight into the molecular interactions between the phytocannabinoid pharmacophore and molecular targets, will help in providing a greater understanding of the physiological role of the endocannabinoid system, and have the potential to be used as new therapeutics.

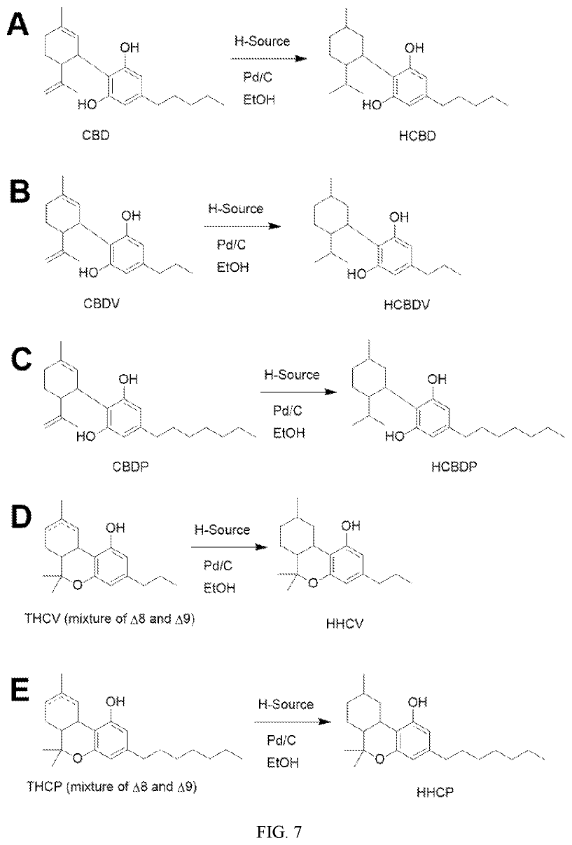

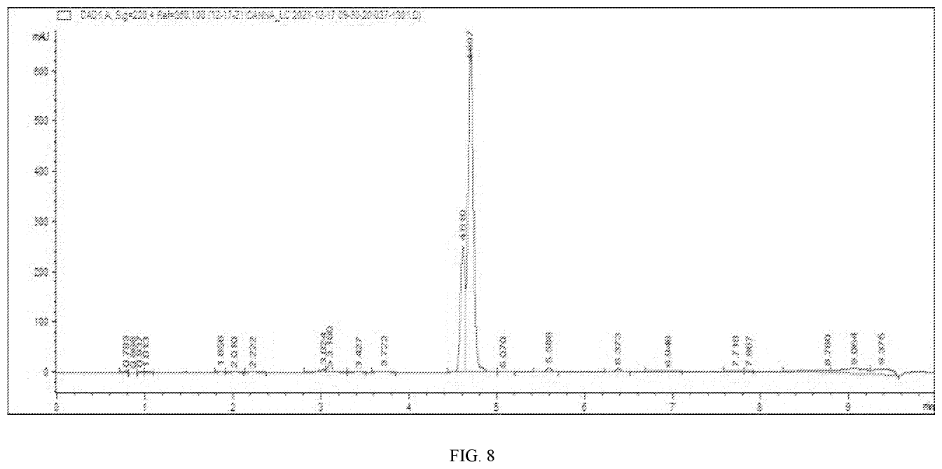

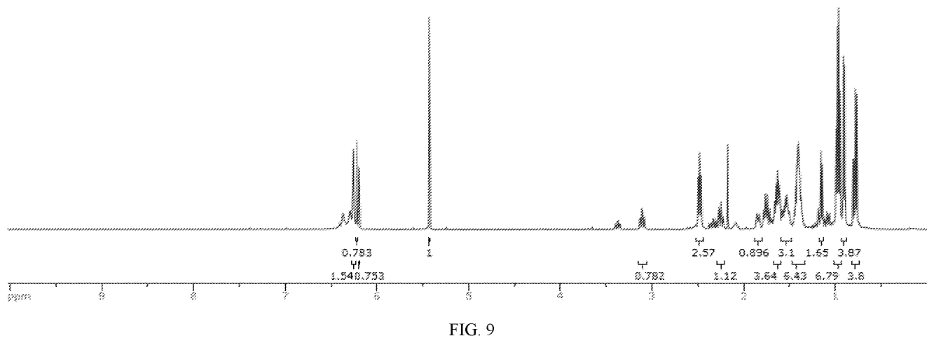

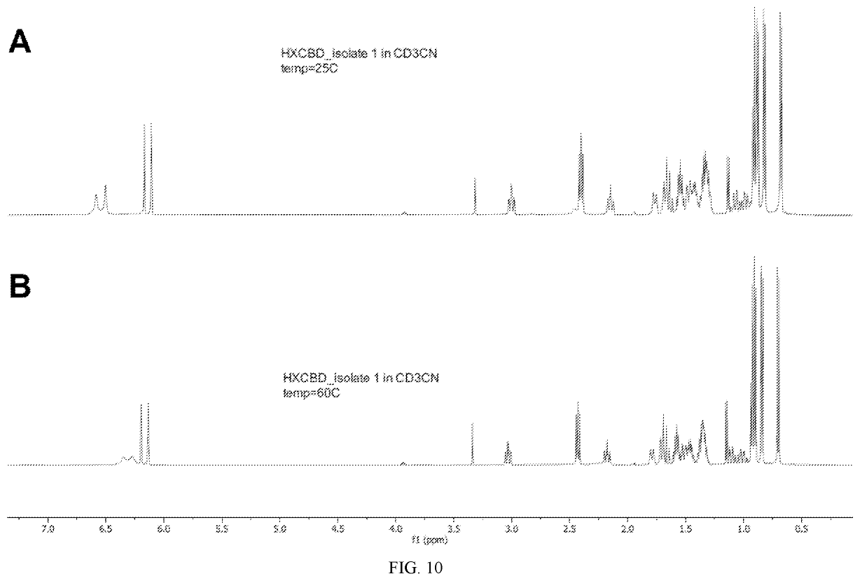

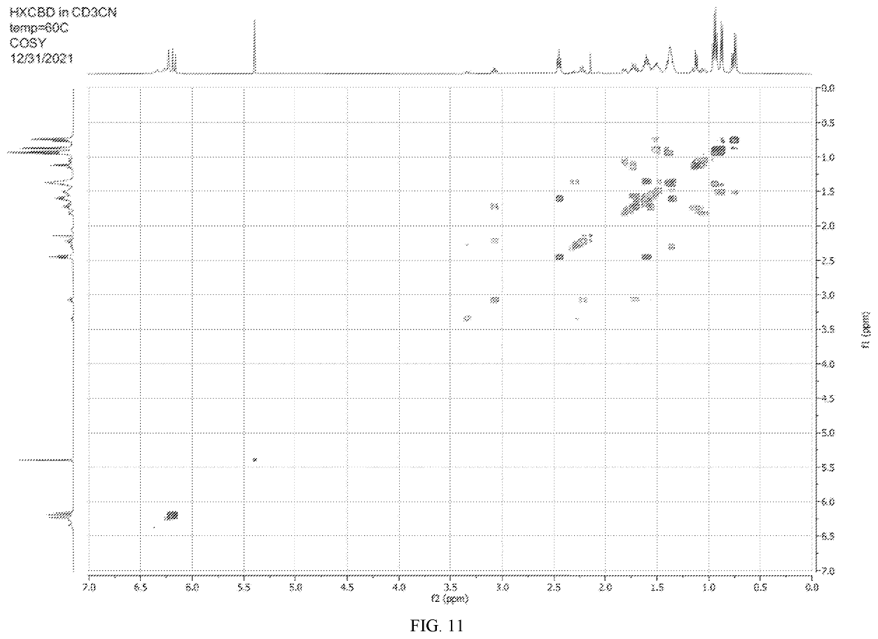

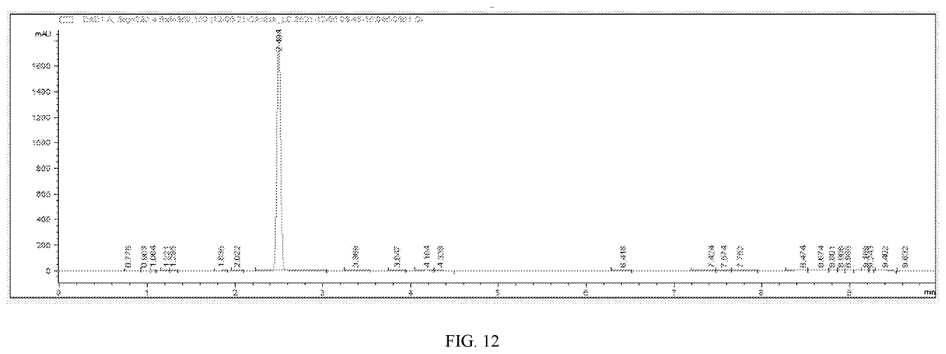

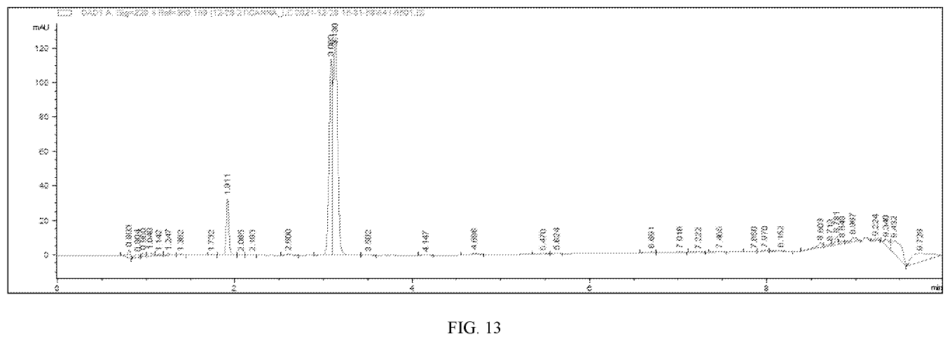

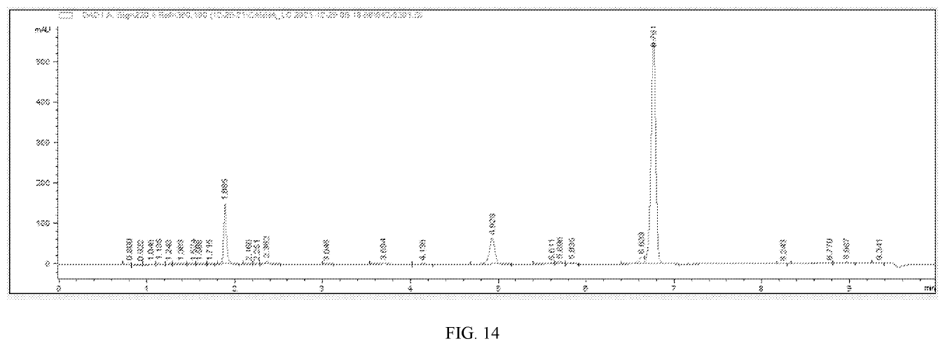

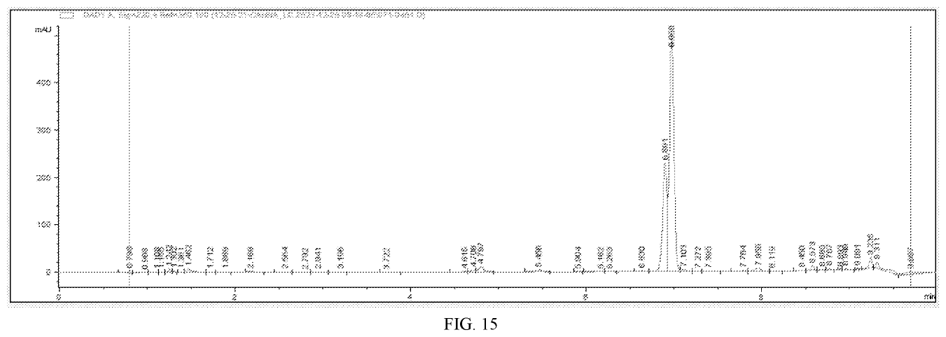

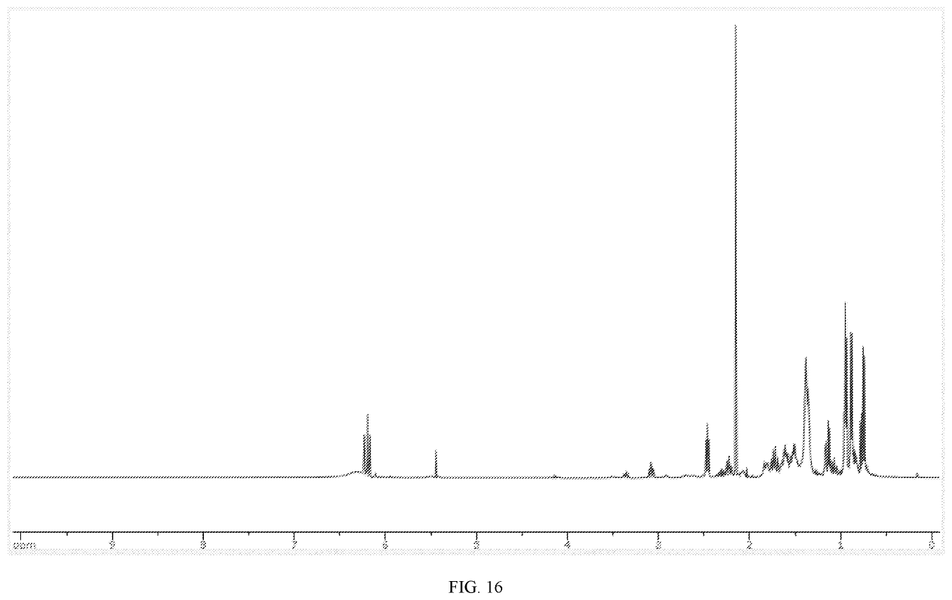

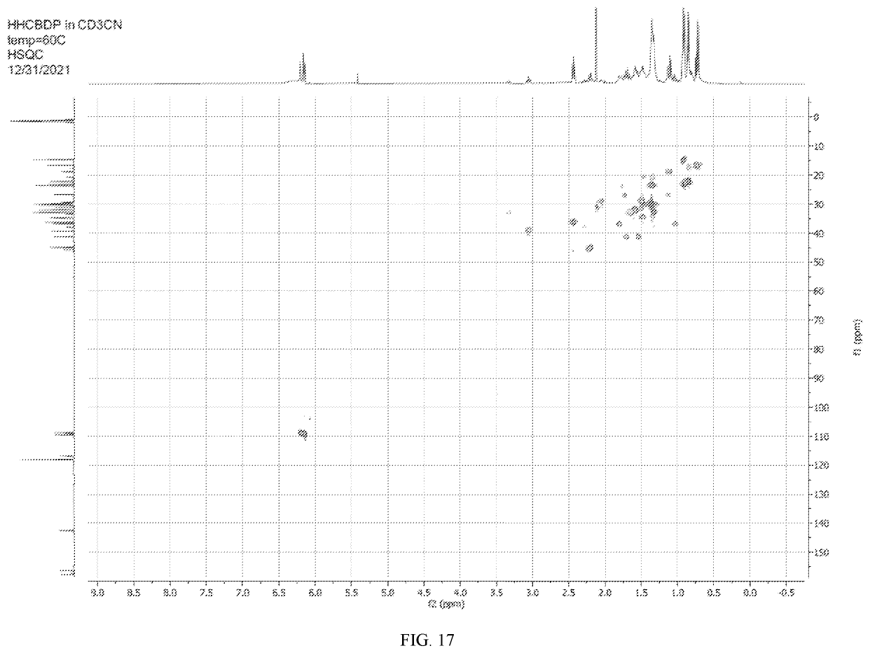

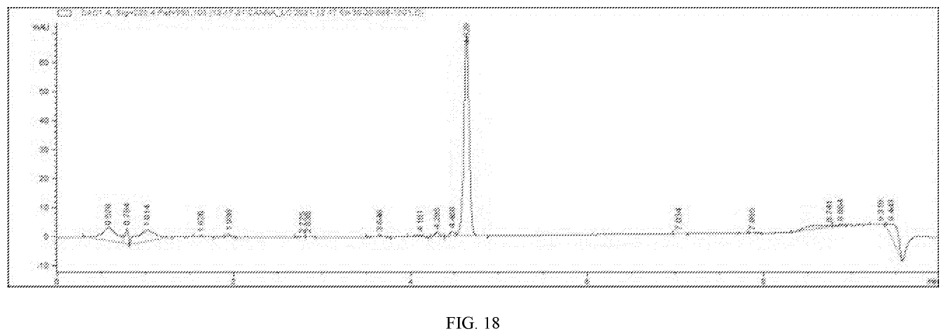

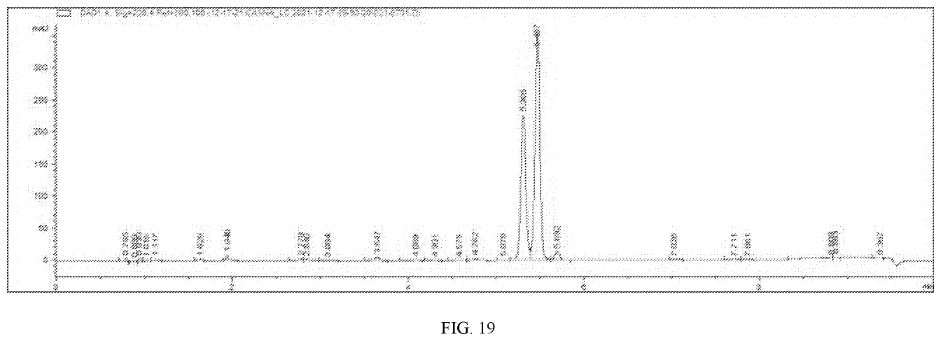

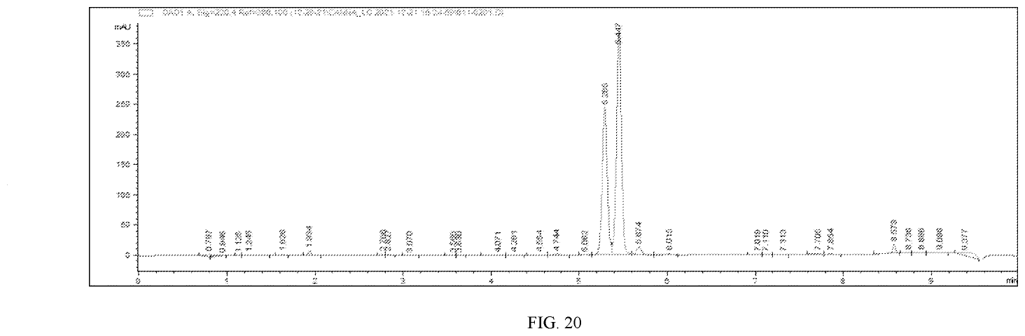

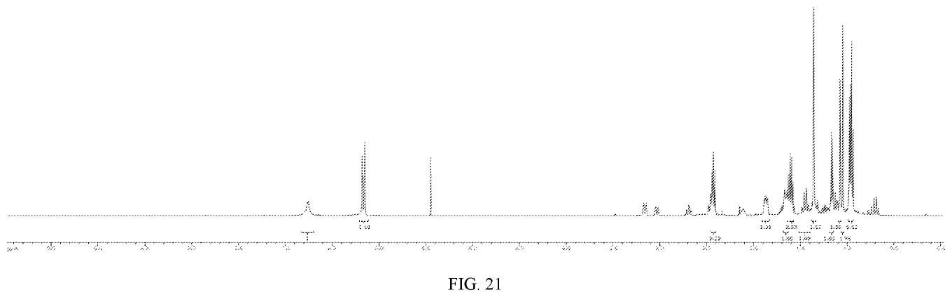

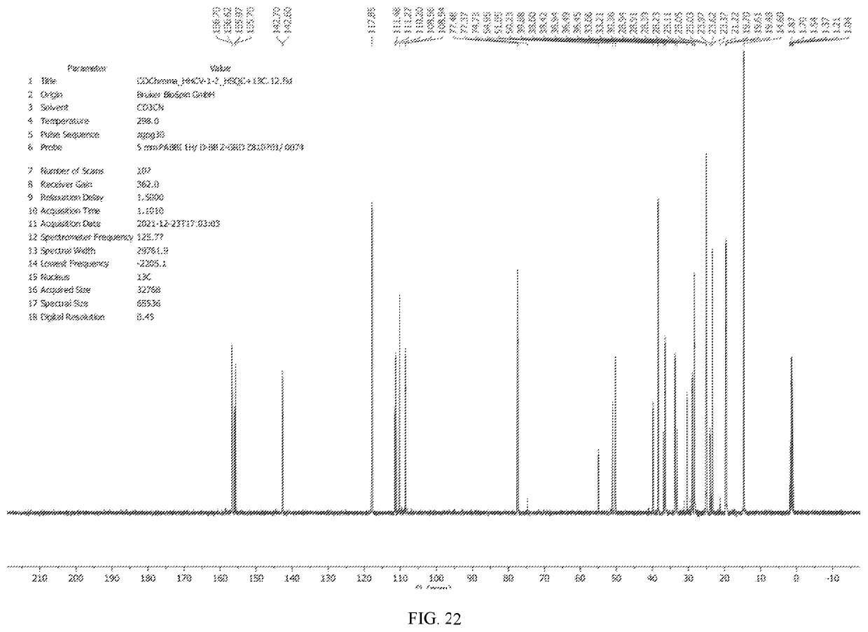

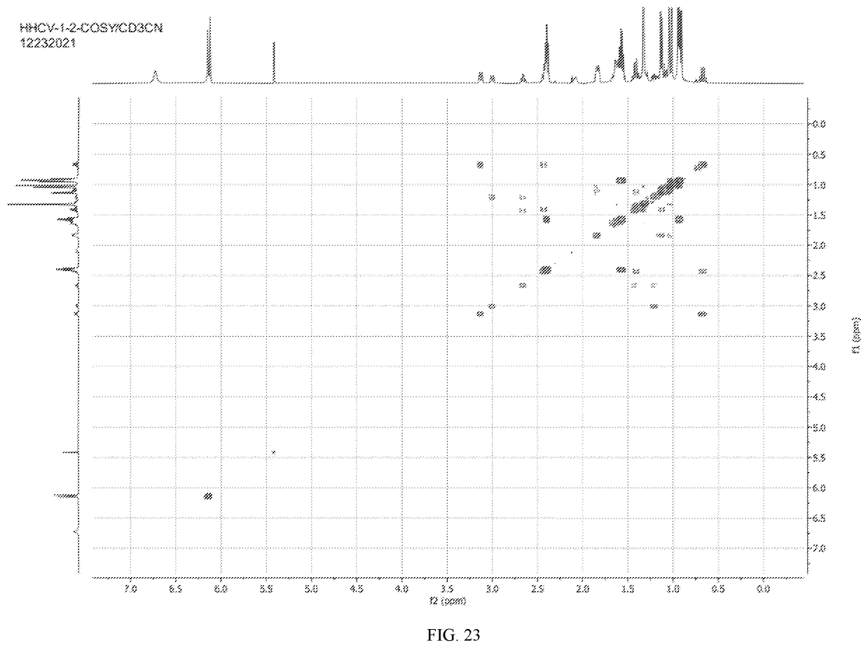

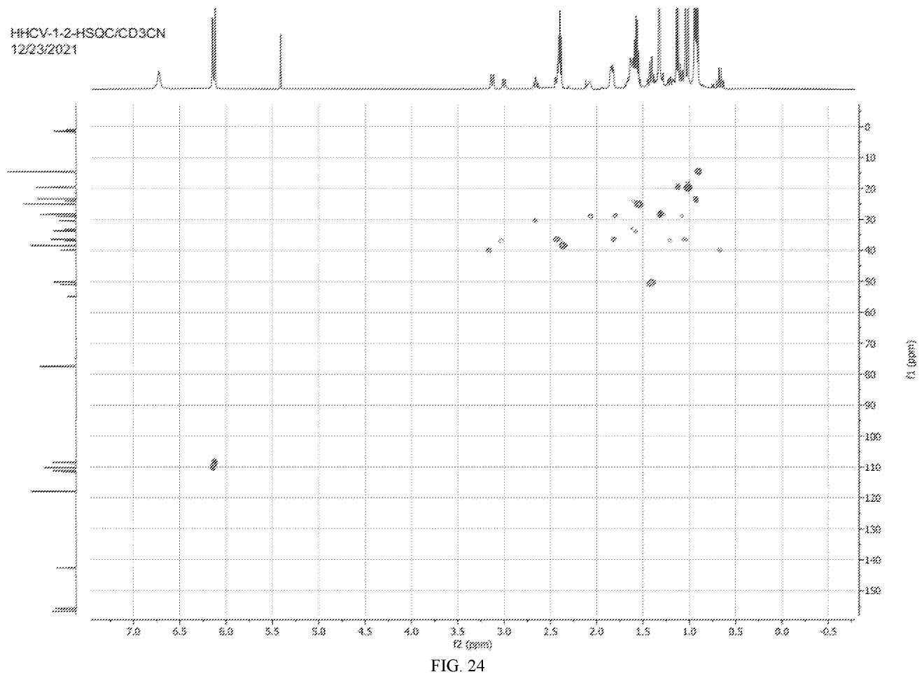

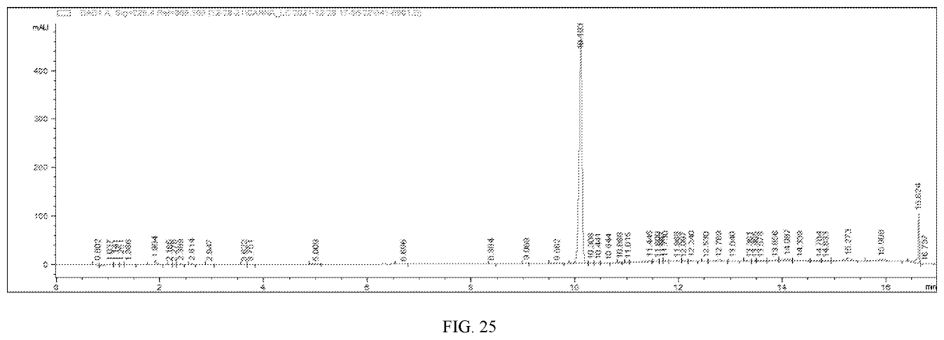

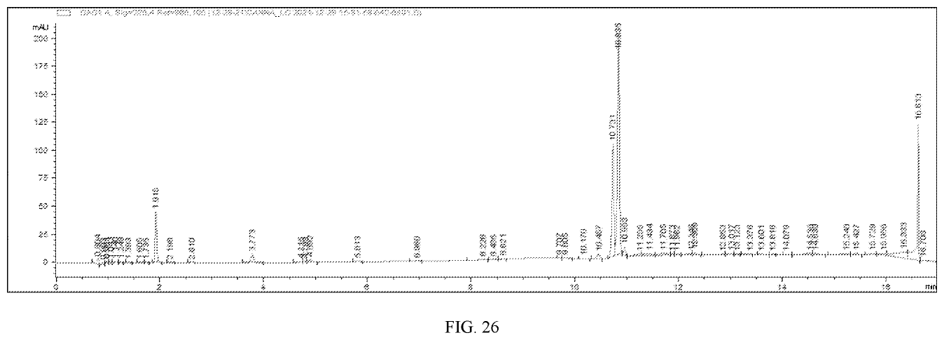

The present inventors have also developed a novel means for synthesizing hexahydrocannabinol from different cannabinoid starting materials. The cannabinoid starting materials include an olefin in the cyclohexenyl group opposite the aromatic ring, i.e., the ring that does not share carbon atoms with the aromatic ring. The olefin may be positioned between any two carbon atoms of the cyclohexenyl group. The method entails hydrogenating the cyclohexenyl olefin to provide hexahydrocannabinol having the corresponding, hydrogenated cyclohexyl group. This single-step method provides a novel means by which hexahydrocannabinol can be synthesized from relatively abundant and inexpensive starting materials. Exemplary reaction schemes for synthesizing hexahydrocannabinol are depicted in FIG. 7. The reactions disclosed herein have been performed safely at large scales (up to 10 kg), and offer a means for producing kilogram-scale amounts of hexahydrocannabinol product.

### Chemical Definitions

The terms delta-8 tetrahydrocannabinol, delta-8 THC, D8 THC, Δ8-tetrahydrocannabinol, and Δ8-THC are used interchangeably herein. The terms delta-9 tetrahydrocannabinol, delta-9 THC, D9 THC, Δ9-tetrahydrocannabinol, Δ9-THC, and THC are used interchangeably herein. The terms hexahydrocannabinol and HHC are used interchangeably herein. The term “semi-synthetic” is defined as a method that employs natural compounds or compounds derived from natural compounds as starting materials to produce different compounds.

The term “alkyl” includes straight-chain alkyl, branched-chain alkyl, cycloalkyl (alicyclic), cyclic alkyl, aryl-unsubstituted alkyl, aryl-substituted alkyl, heteroatom-unsubstituted alkyl, heteroatom-substituted alkyl, heteroatom-unsubstituted Cn-alkyl, and heteroatom-substituted Cn-alkyl. In certain embodiments, lower alkyls are contemplated. In some aspects, the term “alkyl group” denotes a monovalent linear or branched saturated hydrocarbon group of 1 to 12 carbon atoms. In certain embodiments, an alkyl group has 1 to 7 carbon atoms, and in more particular embodiments 1 to 4 carbon atoms. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃) CH₂CH₃ (sec-butyl), —C(CH₃)₂(CH₂)₅CH₃ (dimethylheptyl), —CH₂CH(CH₃)₂ (iso-butyl), —C(CH₃)₃ (tert-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —(CH₂)₂F, —(CH₂)₃F, —(CH₂)₄F, —(CH₂)₅F, —(CH₂)₆F, —(CH₂)₇F, —(CH₂)₈F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃. The term “aryl” refers to a radical, having a single carbon atom as a point of attachment, wherein the carbon atom is part of an aromatic ring structure containing only carbon atoms, 5 or more hydrogen atoms, and no heteroatoms. The -phenyl and -naphthalenyl groups are non-limiting examples of aryl groups. The -benzyl group is a non-limiting example of an aryl-substituted alkyl group, where the alkyl group is methylene —CH₂— and the aryl group is a phenyl group.

The term “olefin” refers to a carbon-carbon double bond. The term “cyclohexyl” denotes a cyclized alkyl group having 6 carbon atoms. The term “cyclohexenyl” denotes a cyclized alkyl group having 6 carbon atoms and further having at least one nonaromatic carbon-carbon double bond. The phrase “cyclohexenyl olefin” refers to a carbon-carbon double bond or olefin of a cyclohexenyl ring.

The term “cyclohexyl” denotes a cyclized alkyl group having 6 carbon atoms. The term “cyclohexenyl” denotes a cyclized alkyl group having 6 carbon atoms and further having at least one nonaromatic carbon-carbon double bond.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Various forms of palladium catalyst useful for the reaction are discussed by Blaser et. al., Supported palladium catalysts for fine chemicals synthesis in Journal of Molecular Catalysis A: Chemical, 2001, v. 172, p. 3-18, the entirety of which is incorporated by reference.

Various forms of palladium catalyst useful for the reaction are discussed by Blaser et. al., Supported palladium catalysts for fine chemicals synthesis in Journal of Molecular Catalysis A: Chemical, 2001, v. 172, p. 3-18, the entirety of which is incorporated by reference.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Scaled Reduction of D8 THC with Hydrogen Gas

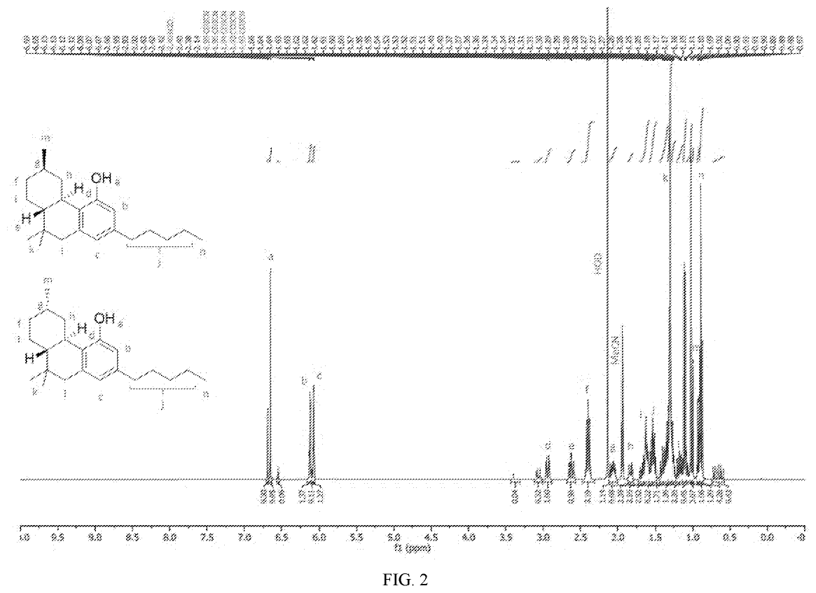

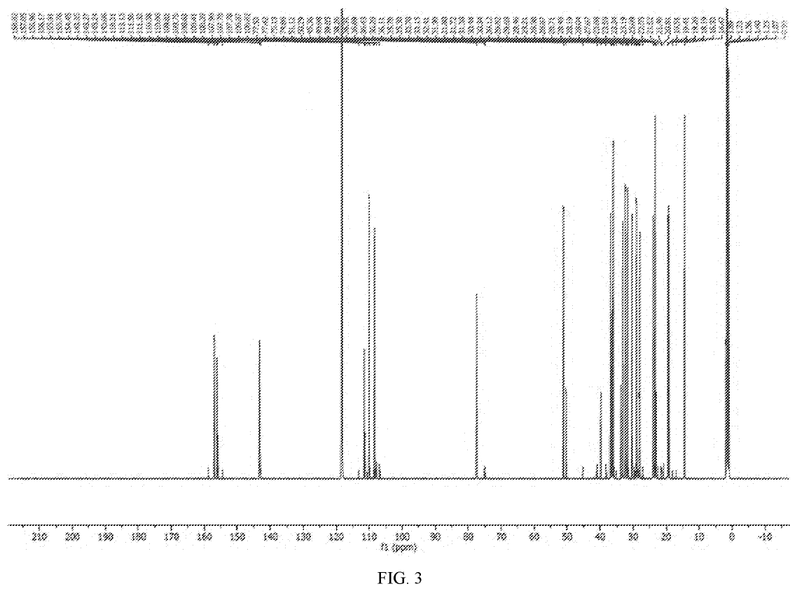

A 200 L reactor equipped with a reflux condenser and an addition funnel was purged with argon for 10-60 minutes at 1-5 bar. Pd/C (0.1 to 5 molar equivalent by percentage of Palladium loading) was added to the reaction slowly using a powder funnel under argon. The reactor is then purged with argon for 10-60 minutes at 1-5 bar. Ethanol (15 to 30 times the mass of starting material) was added slowly so as to avoid sparking the solvent. D8 THC (300 g to KG) was dissolved in minimal amounts of ethanol. The solution is added to the reactor under argon and purged for 10-60 minutes at 1-5 bar. Afterwards, the atmosphere of argon is stopped and an atmosphere of hydrogen (1-5 bar) is introduced. The reaction is then stirred at 25° C. to 50° C. for 3 to 72 hours or until complete by HPLC with a diode array detector. Upon completion, the reaction is purged with argon for 10-60 minutes at 1-5 bar. The reaction mixture is concentrated in vacuo down to less than 50 L and then filtered over 1-3 micron filter paper on a buchner funnel. The solution is then evaporated down the rest of the way. The crude oil is then dissolved in hexane and purified over silica (0 to 5% Ethyl Acetate). The fractions of interest are concentrated in vacuo and then distilled to afford a colorless to light yellow oil with three compounds of similar m/z ratios. 1H NMR spectrum (FIG. 2) of the hexahdyrocannabinol product obtained using a Bruker AVANCE II 500 NMR. ¹H NMR (500 MHz, CD₃CN) δ 0.88, 0.89, 0.91, 0.92, 0.93, 1.00, 1.02, 1.10, 1.12, 1.17, 1.17, 1.18, 1.18, 1.30, 1.31, 1.38, 1.40, 1.42, 1.43, 1.53, 1.62, 1.63, 1.66, 1.83, 1.84, 1.84, 1.84, 1.94, 2.06, 2.27, 2.27, 2.38, 2.40, 2.40, 2.42, 2.60, 2.61, 2.63, 2.63, 2.65, 2.65, 3.06, 3.07, 3.09, 3.09, 6.08, 6.08, 6.12, 6.12, 6.68. ¹³C NMR spectrum (FIG. 3) of the hexahdyrocannabinol product obtained using a Bruker AVANCE II 500 NMR. 13C NMR (126 MHZ, CD₃CN) δ 0.90, 1.07, 1.23, 1.40, 1.56, 1.73, 1.89, 14.47, 19.20, 19.41, 19.51, 23.09, 23.34, 23.88, 28.04, 28.19, 28.87, 28.98, 30.34, 31.72, 32.41, 33.15, 33.70, 36.11, 36.43, 36.91, 39.85, 50.29, 51.12, 77.42, 108.39, 108.41, 110.00, 111.56, 118.31, 143.24, 156.17, 156.96.

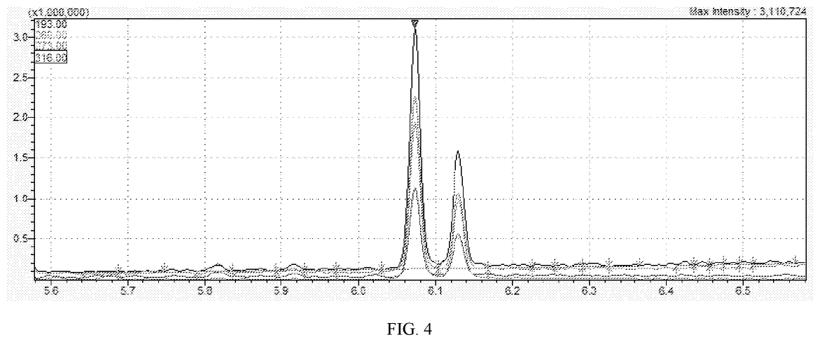

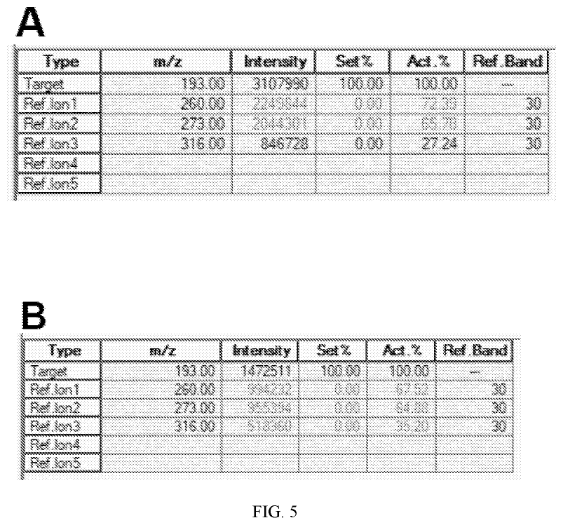

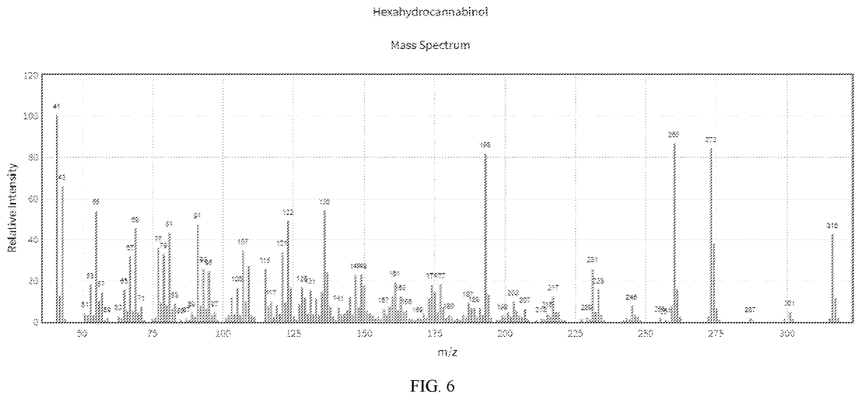

The hexahydrocannabinol product was analyzed using GC/MS. The GC trace is depicted in FIG. 4. The GC trace includes two prominent peaks, and the most prominent ions corresponding to each of the two peaks are included in FIGS. 5A-5B. FIG. 5A includes data corresponding to the left peak of the GC trace, and includes prominent ions at m/z 193, 273, and 260. FIG. 5B includes data corresponding to the right peak of the LC trace, and includes prominent ions at m/z 193, 273, and 260. These data are in agreement with the hexahydrocannabinol mass spectrum included in the National Institute of Standards and Technology (NIST) database (FIG. 6). Like the MS data reported for the hexahydrocannabinol product, the NIST data also includes prominent ions reported at m/z 193, 273, and 260.

### Example 2

### Reduction of D8 THC with Additive that Generates Hydrogen Gas In Situ

To a two-neck round bottomed flask equipped with a reflux condenser, D8 THC (3-50 grams) was weighed and charged with methanol (50-200 mL). The flask is purged of air using vacuum. The flask is then filled with argon. The purge/fill cycle is then repeated three times total. Afterwards slowly add ammonium formate/formic acid (1 to 20 molar equivalents) to the round bottomed flask. The reaction mixture is then heated to 50° C. Once the reaction is at 50° C., add Pd/C (0.1 to 5 molar equivalent by percentage of Palladium loading) to the reaction slowly using a powder funnel. The reaction is then left to stir until completion using HPLC as a guide. Once complete, the reaction mixture is then filtered over celite to remove the Pd/C. The mixture is then placed onto a roto evaporator to remove all methanol. It is then dissolved in hexane. The reaction mixture dissolved in hexane is then washed with water (10-100 mL, 3 times) in a separatory funnel. The aqueous layer is removed after each wash. The organic layer is then washed with a saturated brine solution (10-100 mL) and the aqueous layer is removed. The organic layer is then concentrated in vacuo. This brown oil can then be purified via distillation or chromatography.

### Example 3

### Reduction of D8 THC with Hydrogen Gas and Hydrogen Gas-Generating Additive

To a two-neck round bottomed flask equipped with a reflux condenser, D8 THC (3-50 grams) was weighed and charged with ethanol (50-200 mL). The flask is purged of air using vacuum. The flask is then filled with argon. The purge/fill cycle is then repeated three times total with continuous argon flow after the final cycle. Afterwards slowly add ammonium formate/formic acid (1 to 20 molar equivalents) to the round bottomed flask. Wait 10 minutes then add Pd/C (0.1 to 5 molar equivalent by percentage of Palladium loading) to the reaction slowly using a powder funnel. The atmosphere of argon gas is stopped, and an atmosphere of hydrogen (1-5 bar) is introduced to the reaction flask. The reaction is then left to stir until completion using HPLC as a guide. Once complete, the hydrogen atmosphere is stopped, and the reaction mixture is then filtered over celite to remove the Pd/C. The mixture is then placed onto a roto evaporator to remove all ethanol. It is then dissolved in hexane. The reaction mixture dissolved in hexane is then washed with water (10-100 mL, 3 times) in a separatory funnel. The aqueous layer is removed after each wash. The organic layer is then washed with a saturated brine solution (10-100 mL) and the aqueous layer is removed. The organic layer is then concentrated in vacuo. This brown oil can then be purified via distillation or chromatography. An HPLC trace of a purified HHC product is depicted in FIG. 4.

### Example 4

### Synthesis of HCBD with Hydrogen Gas

A 20 L flask equipped with a reflux condenser and an addition funnel was purged with argon for 10-60 minutes at 1-5 bar. Pd/C (0.1 to 5 molar equivalent by percentage of Palladium loading) was added to the reaction slowly using a powder funnel under argon. The flask was then purged with argon for 10-60 minutes at 1-5 bar. Ethanol (10 to 15 times the mass of starting material) was added slowly so as to avoid sparking the solvent. CBD (100 g) was dissolved in minimal amounts of ethanol. The solution was added to the flask under argon and purged for 10-60 minutes at 1-5 bar. Afterwards, the atmosphere of argon was stopped and an atmosphere of hydrogen (1-5 bar) was introduced. The reaction was then stirred at 25° C. to 50° C. for 3 to 72 hours or until complete by HPLC with a diode array detector. Upon completion, the reaction was purged with argon for 10-60 minutes at 1-5 bar. The reaction mixture was poured over 1-3 micron filter paper on a buchner funnel and then concentrated in vacuo. The crude oil was then dissolved in hexane and purified over silica (0 to 5% Ethyl Acetate). The fractions of interest were concentrated in vacuo and then distilled to afford an orange oil with two compounds. HPLC (C18): 4.610, 4.697 min, ¹H NMR (500 MHZ, CD₃CN) δ 6.25 (2H), 6.21 (1H), 6.19 (1H), 5.42 (1H), 3.10 (1H), 2.48 (3H), 2.25 (1H), 1.84 (1H), 1.62 (4H), 1.54 (3H), 1.4 (6H), 1.15 (2H), 0.96 (7H), 0.91 (4H), 0.78 (4H).

### Example 5

### Synthesis of HCBD Using an Additive

To a two-neck round bottomed flask equipped with a reflux condenser, CBD (2 grams) was added and subsequently charged with methanol (50-300 mL). The flask was purged of air using vacuum. The flask was then filled with argon. The purge/fill cycle was then repeated three times total. Ammonium formate (1 to 20 molar equivalents) was slowly added to the round bottomed flask. Pd/C (0.1 to 5 molar equivalent by percentage of Palladium loading) was added to the reaction slowly using a powder funnel. The reaction was then left to stir until completion using HPLC as a guide. Once complete, the reaction mixture was then filtered over celite to remove the Pd/C. The flask was then placed onto a roto evaporator to remove all methanol. The reaction mixture was then dissolved in hexane and was then washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This orange oil was purified via distillation.

### Example 6

### Synthesis of HCBD Using an Additive and Gas

To a two-neck round bottomed flask equipped with a reflux condenser, D8 THC (2 grams) was added and subsequently charged with ethanol (50-300 mL). The flask was purged of air using vacuum. The flask is then filled with argon. The purge/fill cycle was then repeated three times total with continuous argon flow after the final cycle. Ammonium formate (1 to 20 molar equivalents) was then slowly added to the round bottomed flask. After 10 minutes Pd/C (0.1 to 5 molar equivalent by percentage of Palladium loading) was added to the reaction slowly using a powder funnel. The atmosphere of argon gas was stopped, and an atmosphere of hydrogen (1-5 bar) was introduced to the reaction flask. The reaction was then left to stir until completion using HPLC as a guide. Once complete, the hydrogen atmosphere was stopped, and the reaction mixture was then filtered over celite to remove the Pd/C. The flask was then placed onto a roto evaporator to remove all ethanol. The reaction mixture was dissolved in hexane then washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This orange oil was then purified via distillation.

### Example 7

### Synthesis of HHCV with Hydrogen Gas

A 500 mL flask equipped with a reflux condenser and an addition funnel was purged with argon for 10-15 minutes at 1-5 bar. Pd/C (0.1 to 5 molar equivalent by percentage of Palladium loading) was added to the reaction slowly using a powder funnel under argon. The flask was then purged with argon for 10-60 minutes at 1-5 bar. Ethanol (10 to 15 times the mass of starting material) was added slowly so as to avoid sparking the solvent. A mixture of D9 and D8 THCV (2 g) was dissolved in minimal amounts of ethanol. The solution was added to the flask under argon and purged for 10-15 minutes at 1-5 bar. Afterwards, the atmosphere of argon was stopped and an atmosphere of hydrogen (1-5 bar) was introduced. The reaction was then stirred at 25° C. to 50° C. for 3 to 72 hours or until complete by HPLC with a diode array detector. Upon completion, the reaction was purged with argon for 10-15 minutes at 1-5 bar. The reaction mixture was poured over 1-3 micron filter paper on a buchner funnel and then concentrated in vacuo. The crude oil was then dissolved in hexane and purified over silica (0 to 5% Ethyl Acetate). The fractions of interest were concentrated in vacuo and then distilled to afford an orange oil with two compounds. HPLC (C18): 5.85, 5.447 min, 1H NMR (500 MHZ, CD₃CN) δ 6.75 (1H), 6.167 (2H), 2.44 (3H), 1.86 (1H), 1.61 (3H), 1.44 (1H), 1.35 (4H), 1.15 (2H), 1.07 (2H), 1.06 (2H), 0.95 (6H)

### Example 8

### Synthesis of HHCV Using an Additive

To a two-neck round bottomed flask equipped with a reflux condenser, a mixture of D9 and D8 THCV (2 grams) was added and the flask was subsequently charged with methanol (50-300 mL). The flask was purged of air using vacuum. The flask was then filled with argon. The purge/fill cycle was then repeated three times total. Ammonium formate (1 to 20 molar equivalents) was slowly to the round bottomed flask. Pd/C (0.1 to 5 molar equivalent by percentage of Palladium loading) was added to the reaction slowly using a powder funnel. The reaction was then left to stir until completion using HPLC as a guide. Once complete, the reaction mixture was then filtered over celite to remove the Pd/C. The flask was then placed onto a roto evaporator to remove all methanol. The reaction mixture was dissolved in hexane then washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This orange oil was then purified via distillation.

### Example 9

### Synthesis of HHCV Using an Additive and Gas

To a two-neck round bottomed flask equipped with a reflux condenser was added a mixture of D9 and D8 THCV (2 grams) then the flask was subsequently charged with ethanol (50-300 mL). The flask was purged of air using vacuum. The flask was then filled with argon. The purge/fill cycle was then repeated three times total with continuous argon flow after the final cycle. Afterwards ammonium formate (1 to 20 molar equivalents) was slowly to the round bottomed flask. After 10 minutes Pd/C (0.1 to 5 molar equivalent by percentage of Palladium loading) was slowly added to the reaction using a powder funnel. The atmosphere of argon gas was stopped, and an atmosphere of hydrogen (1-5 bar) was introduced to the reaction flask. The reaction was then left to stir until completion using HPLC as a guide. Once complete, the hydrogen atmosphere was stopped, and the reaction mixture was then filtered over celite to remove the Pd/C. The flask was then placed onto a roto evaporator to remove all ethanol. The reaction mixture was then dissolved in hexane and washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This orange was then purified via chromatography.

### Example 10

### Synthesis of HHCP with Hydrogen Gas

A 500 mL flask equipped with a reflux condenser and an addition funnel and was purged with argon for 10-15 minutes at 1-5 bar. Pd/C (0.1 to 5 molar equivalent by percentage of Palladium loading) was added to the reaction slowly using a powder funnel under argon. The flask was then purged with argon for 10-60 minutes at 1-5 bar. Ethanol (10 to 15 times the mass of starting material) was added slowly so as to avoid sparking the solvent. A mixture of D9 and D8 THCP (2 g) was dissolved in minimal amounts of ethanol. The solution was added to the flask under argon and purged for 10-15 minutes at 1-5 bar. Afterwards, the atmosphere of argon was stopped and an atmosphere of hydrogen (1-5 bar) was introduced. The reaction was then stirred at 25° C. to 50° C. for 3 to 72 hours or until complete by HPLC with a diode array detector. Upon completion, the reaction was purged with argon for 10-15 minutes at 1-5 bar. The reaction mixture was poured over 1-3 micron filter paper on a buchner funnel and then concentrated in vacuo. The crude oil was then dissolved in hexane and purified over silica (0 to 5% Ethyl Acetate). The fractions of interest were concentrated in vacuo and then distilled to afford a brown oil with two compounds, HPLC (C18): 10.731, 10.835 min.

### Example 11

### Synthesis of HHCP Using an Additive

To a two-neck round bottomed flask equipped with a reflux condenser was added a mixture of D9 and D8 THCP (2 grams) and the flask was charged with methanol (50-300 mL). The flask was purged of air using vacuum. The flask was then filled with argon. The purge/fill cycle was then repeated three times total. Ammonium formate (1 to 20 molar equivalents) was slowly added to the round bottomed flask. Pd/C (0.1 to 5 molar equivalent by percentage of Palladium loading) was added to the reaction slowly using a powder funnel. The reaction was then left to stir until completion using HPLC as a guide. Once complete, the reaction mixture was then filtered over celite to remove the Pd/C. The flask was then placed onto a roto evaporator to remove all methanol. The reaction mixture was dissolved in hexane then washed with water (10-100 mL, 3 times) in a separatory funnel. The aqueous layer was removed after each wash. The organic layer was then washed with a saturated brine solution (10-100 mL) and the aqueous layer was removed. The organic layer is then concentrated in vacuo. The resulting brown oil was then purified via distillation.

### Example 12

### Synthesis of HHCP Using an Additive and Gas

To a two-neck round bottomed flask equipped with a reflux condenser was added a mixture of D9 and D8 THCV (2 grams) and the flask was charged with ethanol (50-300 mL). The flask was purged of air using vacuum. The flask was then filled with argon. The purge/fill cycle was then repeated three times total with continuous argon flow after the final cycle. Ammonium formate (1 to 20 molar equivalents) was slowly added to the round bottomed flask. After 10 minutes, Pd/C (0.1 to 5 molar equivalent by percentage of Palladium loading) was added to the reaction slowly using a powder funnel. The atmosphere of argon gas was stopped, and an atmosphere of hydrogen (1-5 bar) was introduced to the reaction flask. The reaction was then left to stir until completion using HPLC as a guide. Once complete, the hydrogen atmosphere was stopped, and the reaction mixture was then filtered over celite to remove the Pd/C. The flask was then placed onto a roto evaporator to remove all ethanol. The reaction mixture was dissolved in hexane then washed with water (10-100 mL, 3 times) in a separatory funnel. The aqueous layer was removed after each wash. The organic layer is then washed with a saturated brine solution (10-100 mL) and the aqueous layer is removed. The organic layer was then concentrated in vacuo. The resulting brown oil was then purified via chromatography.

### Example 13

### Synthesis of HCBDV with Hydrogen Gas

A 500 mL flask was equipped with a reflux condenser and an addition funnel and was purged with argon for 10-15 minutes at 1-5 bar. Pd/C (0.1 to 5 molar equivalent by percentage of Palladium loading) was added to the reaction slowly using a powder funnel under argon. The flask was then purged with argon for 10-60 minutes at 1-5 bar. Ethanol (10 to 15 times the mass of starting material) was added slowly so as to avoid sparking the solvent. A mixture of CBDV (2 g) was dissolved in minimal amounts of ethanol. The solution was added to the flask under argon and purged for 10-15 minutes at 1-5 bar. Afterwards, the atmosphere of argon was stopped and an atmosphere of hydrogen (1-5 bar) was introduced. The reaction was then stirred at 25° C. to 50° C. for 3 to 72 hours or until complete by HPLC with a diode array detector. Upon completion, the reaction was purged with argon for 10-15 minutes at 1-5 bar. The reaction mixture was poured over 1-3 micron filter paper on a buchner funnel and then concentrated in vacuo. The crude oil was then dissolved in hexane and purified over silica (0 to 5% Ethyl Acetate). The fractions of interest are concentrated in vacuo and then distilled to afford a brown oil with two compounds. HPLC (C18): 3.083, 3.139.

### Example 14

### Synthesis of HCBDV Using an Additive

To a two-neck round bottomed flask equipped with a reflux condenser was added CBDV (2 grams) and the flask was charged with methanol (50-300 mL). The flask was purged of air using vacuum. The flask was then filled with argon. The purge/fill cycle was then repeated three times total. Afterwards ammonium formate (1 to 20 molar equivalents) was slowly added to the round bottomed flask. Pd/C (0.1 to 5 molar equivalent by percentage of Palladium loading) was added to the reaction slowly using a powder funnel. The reaction was then left to stir until completion using HPLC as a guide. Once complete, the reaction mixture was then filtered over celite to remove the Pd/C. The flask was then placed onto a roto evaporator to remove all methanol. The reaction mixture was dissolved in hexane then washed with water (10-100 mL, 3 times) in a separatory funnel. The aqueous layer was removed after each wash. The organic layer V then washed with a saturated brine solution (10-100 mL) and the aqueous layer was removed. The organic layer is then concentrated in vacuo. This brown oil was then purified via distillation.

### Example 15

### Synthesis of HCBDV Using an Additive and Gas

To a two-neck round bottomed flask equipped with a reflux condenser was added CBDV (2 grams) and the flask was charged with ethanol (50-300 mL). The flask was purged of air using vacuum. The flask was then filled with argon. The purge/fill cycle was then repeated three times total with continuous argon flow after the final cycle. Afterwards ammonium formate (1 to 20 molar equivalents) was slowly added to the round bottomed flask. After 10 minutes Pd/C (0.1 to 5 molar equivalent by percentage of Palladium loading) was added to the reaction slowly using a powder funnel. The atmosphere of argon gas was stopped, and an atmosphere of hydrogen (1-5 bar) was introduced to the reaction flask. The reaction was then left to stir until completion using HPLC as a guide. Once complete, the hydrogen atmosphere was stopped, and the reaction mixture was then filtered over celite to remove the Pd/C. The flask was then placed onto a roto evaporator to remove all ethanol. The reaction mixture was then dissolved in hexane then washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This brown oil was purified via chromatography.

### Example 16

### Synthesis of HCBDP with Hydrogen Gas

A 500 mL flask equipped with a reflux condenser and an addition funnel was purged with argon for 10-15 minutes at 1-5 bar. Pd/C (0.1 to 5 molar equivalent by percentage of Palladium loading) was added to the reaction slowly using a powder funnel under argon. The flask was then purged with argon for 10-60 minutes at 1-5 bar. Ethanol (10 to 15 times the mass of starting material) was added slowly so as to avoid sparking the solvent. CBDP (2 g) was dissolved in minimal amounts of ethanol. The solution was added to the flask under argon and purged for 10-15 minutes at 1-5 bar. Afterwards, the atmosphere of argon was stopped and an atmosphere of hydrogen (1-5 bar) was introduced. The reaction was then stirred at 25° C. to 50° C. for 3 to 72 hours or until complete by HPLC with a diode array detector. Upon completion, the reaction was purged with argon for 10-15 minutes at 1-5 bar. The reaction mixture was poured over 1-3 micron filter paper on a buchner funnel and then concentrated in vacuo. The crude oil was then dissolved in hexane and purified over silica (0 to 5% Ethyl Acetate). The fractions of interest are concentrated in vacuo and then distilled to afford a brown oil with two compounds. HPLC (C18): 6.891, 6.968 min.

### Example 17

### ACCE Synthesis of HCBDP Using an Additive

To a two-neck round bottomed flask equipped with a reflux condenser was added CBDP (2 grams) and the flask was charged with methanol (50-300 mL). The flask was purged of air using vacuum. The flask was then filled with argon. The purge/fill cycle was then repeated three times total. Afterwards ammonium formate (1 to 20 molar equivalents) was slowly added to the round bottomed flask. Pd/C (0.1 to 5 molar equivalent by percentage of Palladium loading) was added to the reaction slowly using a powder funnel. The reaction was then left to stir until complete using HPLC as a guide. Once complete, the reaction mixture was then filtered over celite to remove the Pd/C. The flask was then placed onto a roto evaporator to remove all methanol. The reaction mixture was dissolved in hexane then washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This brown oil was then purified via distillation.

### Example 18

### Synthesis of HCBDP Using an Additive and Gas

To a two-neck round bottomed flask equipped with a reflux condenser was added CBDP (2 grams) and the flask was charged with ethanol (50-300 mL). The flask was purged of air using vacuum. The flask was then filled with argon. The purge/fill cycle was then repeated three times total with continuous argon flow after the final cycle. Afterwards ammonium formate (1 to 20 molar equivalents) was slowly added to the round bottomed flask. After 10 minutes Pd/C (0.1 to 5 molar equivalent by percentage of Palladium loading) was added slowly to the reaction using a powder funnel. The atmosphere of argon gas was stopped, and an atmosphere of hydrogen (1-5 bar) was introduced to the reaction flask. The reaction was then left to stir until complete using HPLC as a guide. Once complete, the hydrogen atmosphere was stopped, and the reaction mixture was then filtered over celite to remove the Pd/C. The flask was then placed onto a roto evaporator to remove all ethanol. The reaction mixture was dissolved in hexane then washed with water (10-100 mL, 3 times) in a separatory funnel. The aqueous layer was removed after each wash. The organic layer was then washed with a saturated brine solution (10-100 mL) and the aqueous layer was removed. The organic layer was then concentrated in vacuo. This brown oil was then purified via chromatography.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for producing hexahydrocannabinol, comprising:
providing a starting composition comprising delta-8 tetrahydrocannabinol, delta-9 tetrahydrocannabinol, or a mixture thereof to a reaction vessel;
providing a catalyst to the reaction vessel;
providing hydrogen gas, a source of hydrogen gas, or a combination thereof to the reaction vessel;
heating the reaction vessel to a temperature sufficient to effect hydrogenation of the delta-8 tetrahydrocannabinol, delta-9 tetrahydrocannabinol, or mixture thereof;
wherein hydrogenation of the delta-8 tetrahydrocannabinol, delta-9 tetrahydrocannabinol, or mixture thereof produces hexahydrocannabinol.

2. The method of claim 1, wherein the catalyst is provided in an amount ranging from 0.01 to 10 molar equivalents.

3. (canceled)

4. The method of claim 1, wherein the catalyst is selected from the group consisting of Pd/C, Rh/C, Pt/C, Ru/C, Raney nickel. palladium on alumina, palladium on activated charcoal, Pt₂O, [C₈H₁₂IrP(C₆H₁₁)₃C₅H₅N]PF₆, and [RhCl(PPh₃)₃].

5. The method of claim 1, wherein the hydrogen gas is provided in an amount that affords an intra-vessel gas pressure ranging from 1 bar to 20 bar.

6. (canceled)

7. (canceled)

8. The method of claim 1, wherein the source of hydrogen gas comprises ammonium formate and formic acid.

9. The method of claim 8, wherein an amount of ammonium formate ranges from 1 to 40 molar equivalents.

10. The method of claim 8, wherein an amount of formic acid ranges from 1 to 40 molar equivalents.

11. The method of claim 1, further comprising providing a solvent to the reaction vessel prior to the heating step, wherein the solvent is selected from the group consisting of ethanol, methanol, propanol, isopropanol, butanol, sec-butanol, and isobutanol.

12-15. (canceled)

16. The method of claim 1, wherein the heating step comprises heating the reaction vessel to a temperature ranging from 25° C. to 100° C.

17-22. (canceled)

23. A process for the preparation of a hexahydrocannabidiol derivative, comprising:
providing a tetrahydrocannabidiol derivative of formula I to a reaction vessel; 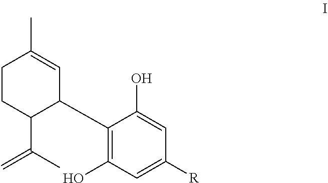
wherein R is hydrogen or a substituted or unsubstituted alkyl group ranging from 1 to 9 carbon atoms;
providing a catalyst to the reaction vessel;
providing hydrogen gas, a source of hydrogen gas, or a combination thereof to the reaction vessel; and
heating the reaction vessel to a temperature sufficient to effect hydrogenation of the cyclohexenyl olefin group to produce a hexahydrocannabidiol derivative of formula II

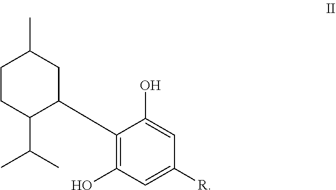

24. The process of claim 23, wherein R is propyl or heptyl.

25-39. (canceled)

40. A process for the preparation of a hexahydrocannabinoid derivative, comprising:
providing a tetrahydrocannabinoid derivative of formula III to a reaction vessel; 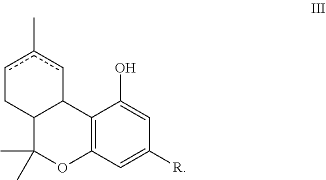
wherein R is hydrogen or a substituted or unsubstituted alkyl group ranging from 1 to 9 carbon atoms;
providing a catalyst to the reaction vessel;
providing hydrogen gas, a source of hydrogen gas, or a combination thereof to the reaction vessel; and
heating the reaction vessel to a temperature sufficient to effect hydrogenation of the cyclohexenyl olefin group to produce a hexahydrocannabinoid derivative of formula IV

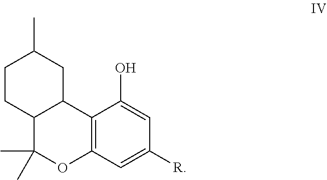

41. (canceled)

42. The process of claim 40, wherein the catalyst is provided in an amount ranging from 0.01 to 10 molar equivalents.

43. The process of claim 42, wherein the catalyst is selected from the group consisting of Pd/C, Pt/C, Rh/C, Ru/C, Raney nickel, Pd/alumina, Pd/activated charcoal, Pt/alumina, Pt/activated charcoal, Pt₂O (Adam's catalyst), Wilkinson's catalyst ([RhCl(PPh₃)₃]), Crabtree's catalyst ([C₈H₁₂IrP(C₆H₁₁)₃C₅H₅N]PF₆), 9-borabicyclo[3.3.1]nonane, alpine borane, BH₃-DMSO, BH₃-THF, and N-methylimidodiacetic (MIDA) boronates.

44. (canceled)

45. The process of claim 40, wherein the hydrogen gas is provided in an amount that affords an intra-vessel gas pressure ranging from 1 bar to 20 bar.

46. (canceled)

47. (canceled)

48. The process of claim 40, wherein the source of hydrogen gas comprises ammonium formate and/or formic acid.

49. The process of claim 48, wherein an amount of ammonium formate ranges from 1 to 40 molar equivalents.

50. The process of claim 48, wherein an amount of formic acid ranges from 1 to 40 molar equivalents.

51. The process of claim 40, further comprising providing a solvent to the reaction vessel prior to the heating step, wherein the solvent is selected from the group consisting of methanol, ethanol, propanol, isopropanol, butanol, tert-butanol, THF, 2-Me-THE, toluene, and ethyl acetate.

52. (canceled)

53. The process of claim 40, wherein the heating step comprises heating the reaction vessel to a temperature ranging from 25° C. to 100° C.

54-67. (canceled)
